(19) United States
(12) Patent Application Publication
(10) Pub. No.: US 2024/0109774 A1
(43) Pub. Date: Apr. 4, 2024

(54) MULTIPLE FURNACE CARBON CAPTURE THROUGH FUEL GAS SEPARATION AND HYDROGEN COMBUSTION PRODUCT ELECTROLYSIS

(71) Applicant: Chevron Phillips Chemical Company LP, The Woodlands, TX (US)

(72) Inventors: James Hillier, Kingwood, TX (US); Ralph J. Price, Porter, TX (US); Arthur Orscheln, Lake Jackson, TX (US)

(21) Appl. No.: 17/937,081

(22) Filed: Sep. 30, 2022

(51) Int. Cl. C01B 3/36; C01B 3/22; C01B 3/50; C01B 3/52; C10G 9/14; C10L 3/10; C25B 1/04

(52) U.S. Cl. CPC C01B 3/36; C01B 3/22; C01B 3/50; C01B 3/52; C10G 9/14; C10L 3/10; C25B 1/04; C01B 2203/0415; C01B 2203/0475; C01B 2203/0495; C01B 2203/062; C01B 2203/0822; C01B 2203/0827; C01B 2203/1241; C01B 2203/145; C01B 2203/148; C01B 2203/86; C10G 2300/4081

(57) ABSTRACT

A hydrogen-rich hydrocarbon fuel gas can be separated into a methane fuel stream and a hydrogen product stream. The methane fuel stream can be fed to a methane fuel fired furnace, combustion of the methane fuel stream can produce a carbon-dioxide-rich flue gas, and a carbon capture process can be performed on the carbon-dioxide-rich flue gas. The hydrogen product stream can be fed to a hydrogen fired furnace or elsewhere. Combustion of the hydrogen product stream in a hydrogen fired furnace can generate a flue gas the is low in carbon dioxide. Electrolysis of water obtained from the hydrogen fired furnace flue gas can produce hydrogen for a desired use, such as fuel for the hydrogen fired furnace, and can produce oxygen for enriching the fuel gas fed to the methane fuel fired furnace.

### FIELD OF THE DISCLOSURE

The present disclosure relates to combustion of a fuel gas in a furnace, to the fuel gas that is fed to the furnace, and to the processing of flue gas emitted from the furnace.

### BACKGROUND

Furnaces are used in the petrochemical industry as well as other industries to supply heat for a particular application. In the petrochemical industry, an exemplary use of furnaces is in a cracking unit to produce an olefin from an alkane, such as ethylene from ethane. The heat in a furnace can be supplied by the combustion of a fuel gas that can contain hydrocarbons, and combustion of hydrocarbons in the furnace can produce a flue gas that contains carbon dioxide and water.

In an effort to reduce carbon dioxide emissions for these types of furnaces, the flue gas emitted from a furnace can be introduced to a carbon capture process, in which carbon dioxide is removed from the flue gas to produce a carbon dioxide product having a high concentration of carbon dioxide and a residual gas product that has a low concentration of carbon dioxide. The residual gas product can then be used or processed accordingly with reduced concern for carbon dioxide emissions.

There is a need to improve furnace operation while maintaining reduced carbon dioxide emissions.

### SUMMARY

Disclosed is a method that can include separating a hydrogen-rich hydrocarbon fuel stream into a hydrogen product stream and a methane fuel stream, wherein the hydrogen-rich hydrocarbon fuel stream comprises hydrogen, methane, and other light hydrocarbons, wherein hydrogen is present in the hydrogen-rich hydrocarbon fuel stream in a range of from about 40 vol % to about 100 vol %; alternatively, in a range of from about 40 vol % to about 90 vol %; alternatively, in a range of from about 90 vol % to about 100 vol % based on a total volume of the hydrogen-rich hydrocarbon fuel stream; combusting the methane fuel stream in a methane fuel fired furnace to produce a flue gas stream comprising carbon dioxide, nitrogen, and water vapor; and feeding the flue gas stream to a carbon capture process.

Disclosed is a hydrocarbon cracking system that can include a methane fuel fired furnace fluidly coupled to a methane fuel stream and a first hydrocarbon feed stream and configured to produce a first hydrocarbon product stream from the first hydrocarbon feed stream and a flue gas from the first fuel gas stream; a first separator fluidly coupled to the first hydrocarbon product stream and configured to separate the first hydrocarbon product stream into a hydrogen-rich hydrocarbon fuel stream and a third hydrocarbon product stream; and a second separator fluidly coupled to the hydrogen-rich hydrocarbon fuel stream and configured to separate the hydrogen-rich hydrocarbon fuel stream into a hydrogen product stream and the methane fuel stream; wherein the methane fuel fired furnace is configured to receive the methane fuel stream.

Disclosed is a method that can include separating a hydrogen combustion product stream into a water stream and a residual gas stream; separating the water stream into a hydrogen fuel stream and an O2 product stream using an electrolyzer; feeding the O2 product stream to a methane fuel fired furnace; and feeding the hydrogen fuel stream to a hydrogen fired furnace.

Disclosed is a hydrocarbon cracking system that can include a methane fuel fired furnace fluidly coupled to a first fuel gas stream and a first hydrocarbon feed stream and configured to produce a first hydrocarbon product stream from the first hydrocarbon feed stream and a flue gas from the first fuel gas stream; a hydrogen fired furnace fluidly coupled to a second fuel gas stream and a second hydrocarbon feed stream and configured to produce a second hydrocarbon product stream from the second hydrocarbon feed stream and a hydrogen combustion product stream from the second fuel gas stream; a first separator fluidly coupled to the hydrocarbon combustion product stream and configured to receive the hydrogen combustion product stream, cool the hydrogen combustion product stream, and separate the cooled hydrogen combustion product stream into a water stream and a residual gas stream; an electrolyzer fluidly coupled to the water stream and configured to separate water received from the water stream into oxygen in an O2 product stream and hydrogen in a hydrogen product stream. The methane fuel fired furnace can be fluidly coupled to the O2 product stream. The hydrogen fired furnace can be fluidly coupled to the hydrogen product stream.

Other technical features may be readily apparent to one skilled in the art from the following figures, descriptions and claims.

### BRIEF DESCRIPTION OF THE DRAWINGS

For a more complete understanding of this disclosure, reference is now made to the following description, taken in conjunction with the accompanying drawings, in which:

FIG. 1 illustrates a schematic diagram of an embodiment of a hydrocarbon cracking system.

FIG. 2 illustrates a schematic diagram of an alternative embodiment of a hydrocarbon cracking system.

FIG. 3 illustrates a schematic diagram of an alternative embodiment of a hydrocarbon cracking system.

FIG. 4 illustrates a schematic diagram of an alternative embodiment of a hydrocarbon cracking system.

FIG. 5 illustrates a schematic diagram of an alternative embodiment of a hydrocarbon cracking system.

FIG. 6 illustrates a schematic diagram of an alternative embodiment of a hydrocarbon cracking system.

FIG. 7 illustrates a schematic diagram of an alternative embodiment of a hydrocarbon cracking system.

### DETAILED DESCRIPTION

Illustrative aspects of the subject matter claimed herein will now be disclosed. In the interest of clarity, not all features of an actual implementation are described in this specification. It can be appreciated that in the development of any such actual aspect, numerous implementation-specific decisions must be made to achieve the developers' specific goals, such as compliance with system-related and business-related constraints, which can vary from one implementation to another. Moreover, it can be appreciated that such a development effort, even if complex and time-consuming, would be a routine undertaking for those of ordinary skill in the art having the benefit of this disclosure.

The term “stream” as used herein refers to a composition of the components disclosed herein for the respective stream. The term “stream” can additionally refer to and imply associated equipment, such as conduit, line, and pipe that is used to move the composition from one location to another (e.g., a stream from one equipment unit to another equipment unit).

The term “conduit” as used herein refers to a tubular structure through which a fluid can flow and having a wall thickness rated for the fluid pressure. A conduit can be embodied as a pipe or tube, for example. Additionally, conduit may refer to a segment of pipe or tubes, or to a series or string of pipes or tubes.

A hydrocarbon-containing fuel gas that is fed to a furnace, such as a furnace in a hydrocarbon cracking unit, can contain hydrogen. It has been found that the presence of hydrogen and hydrogen combustion products in the hydrocarbon-containing fuel gas can reduce the efficiency of downstream carbon capture process(es) that removes the carbon dioxide from the flue gas that is produced by the furnace. The presence of hydrogen in the fuel gas contributes to a reduced efficiency of carbon capture per volume of flue gas because a reduced concentration of carbon dioxide in the flue gas is emitted from the furnace. The carbon capture process thus captures a lower volume of carbon dioxide per volume of flue gas processed by the carbon capture process.

The disclosed processes and systems can separate the hydrogen from the hydrocarbon-containing fuel gas prior to feeding the hydrogen-depleted fuel gas to a methane fuel fired furnace(s). The disclosed processes and systems can also utilize one or more hydrogen fired furnace in combination with one or more methane fuel fired furnace. The hydrogen fired furnace(s) can produce a flue gas (referred to herein also as a hydrogen combustion product stream), and the methane fuel fired furnace(s) can produce a flue gas, where the flue gas of the hydrogen fired furnace(s) has a low concentration of carbon dioxide relative to a concentration of carbon dioxide in the flue gas of the methane fuel fired furnace(s). The relatively high concentration of carbon dioxide in the flue gas of the methane fuel fired furnace(s) allows the carbon capture process(es) that receive the flue gas to operate more efficiently. The disclosed processes and systems can also capture water from the hydrogen combustion product stream, and can subject the water to electrolysis. Electrolysis of the water can produce an O2 product stream and a hydrogen product stream. The O2 product stream can enrich the air that is fed to the methane fuel fired furnace with oxygen, and the hydrogen product stream can supplement the hydrogen-containing fuel gas that is fed to the hydrogen fired furnace.

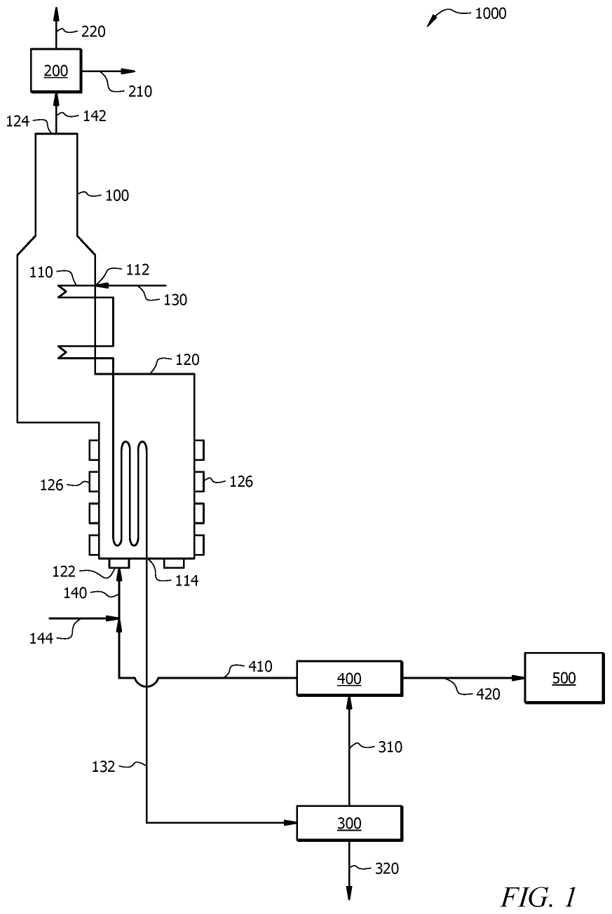

FIGS. 1 to 7 illustrate schematic diagrams of embodiments of hydrocarbon cracking systems 1000, 2000, 3000, 4000, 5000, 6000, and 7000.

The configuration of the hydrocarbon cracking systems 1000, 2000, 3000, 4000, 5000, 6000, and 7000 separate a hydrogen-rich hydrocarbon fuel gas into 1) a methane fuel stream that is directed to a methane fuel fired furnace and 2) a hydrogen product stream that is directed to a unit that consumes or stores hydrogen. The methane fuel stream has a lower concentration of hydrogen than the hydrogen-rich hydrocarbon fuel gas, and the flue gas emitted from the methane fuel fired furnace has a higher concentration of carbon dioxide, compared to a flue gas generated by feeding the hydrogen-rich hydrocarbon fuel gas directly to the methane fuel fired furnace. The higher concentration of carbon dioxide in the flue gas leads to more efficient operation of the carbon capture process compared to carbon capture on a flue gas having a lower concentration of carbon dioxide.

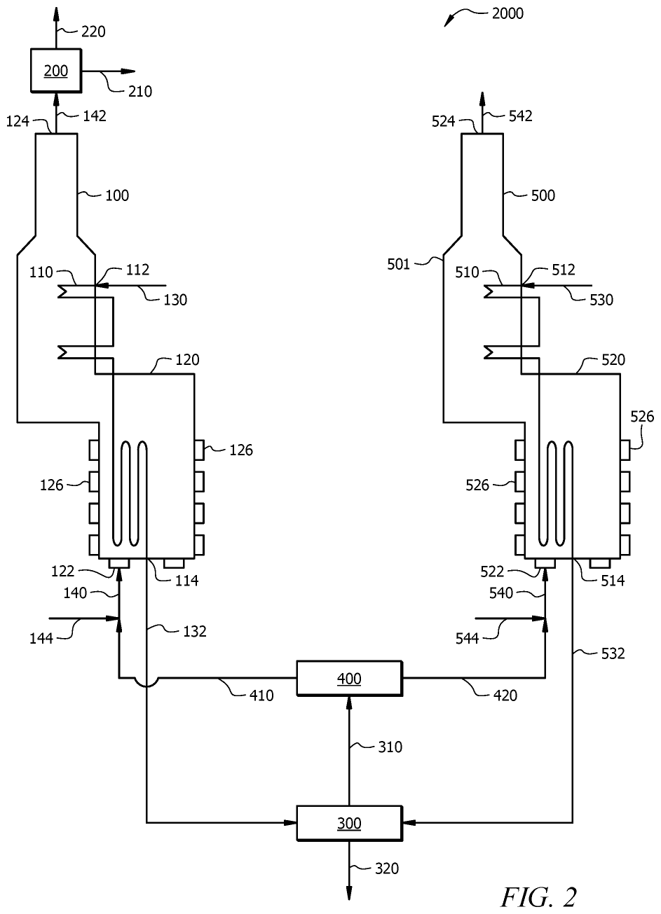

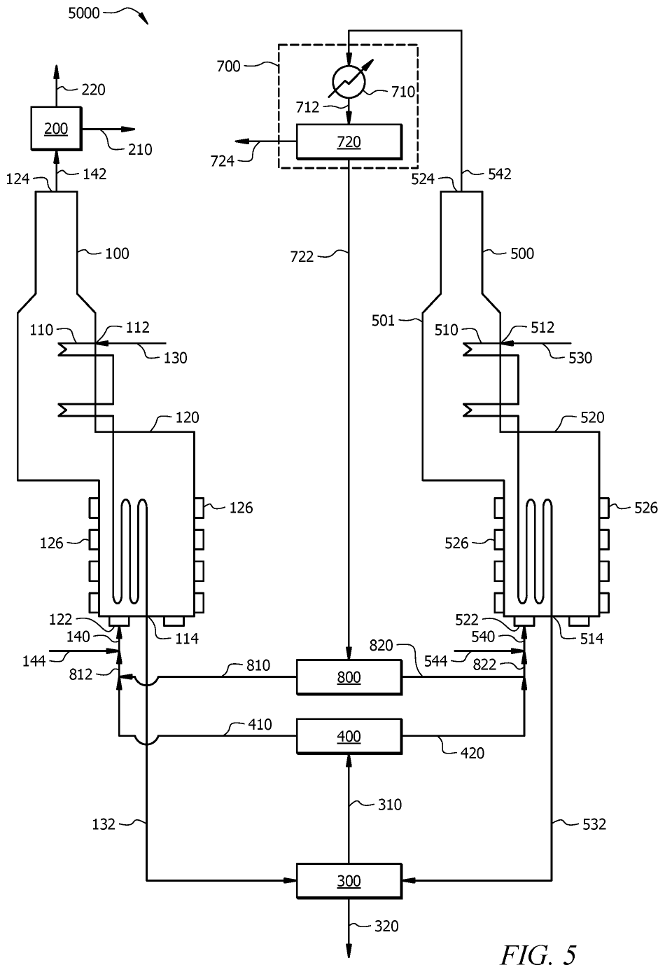

The hydrocarbon cracking systems 2000 and 5000 in FIGS. 2 and 5 illustrate that the unit that receives the hydrogen product stream can be a hydrogen fired furnace. Hydrocarbon cracking systems 2000 and 5000 demonstrate the operation of a methane fuel fired furnace in combination with a hydrogen fired furnace. The methane fuel stream has a lower concentration of hydrogen than the hydrogen-rich hydrocarbon fuel gas, and the flue gas emitted from the methane fuel fired furnace has a higher concentration of carbon dioxide, compared to a flue gas generated by feeding the hydrogen-rich hydrocarbon fuel gas directly to the methane fuel fired furnace. The higher concentration of carbon dioxide in the flue gas leads to more efficient operation of the carbon capture process compared to carbon capture on a flue gas having a lower concentration of carbon dioxide. Additionally, the flue gas produced by the hydrogen fired furnace, also referred to herein as the hydrogen combustion product stream, can be free of carbon dioxide; thus, systems 2000 and 5000 and the processes implemented therein effectively concentrate carbon dioxide in the flue gas of the methane fuel fired furnace while producing a hydrogen combustion product that is free of carbon dioxide. Concentrating the generated carbon dioxide in the fuel gas of the methane fuel fired furnace allows for carbon capture processing of the volume of fuel gas produced while not requiring carbon capture processing of the volume of hydrogen combustion product produced.

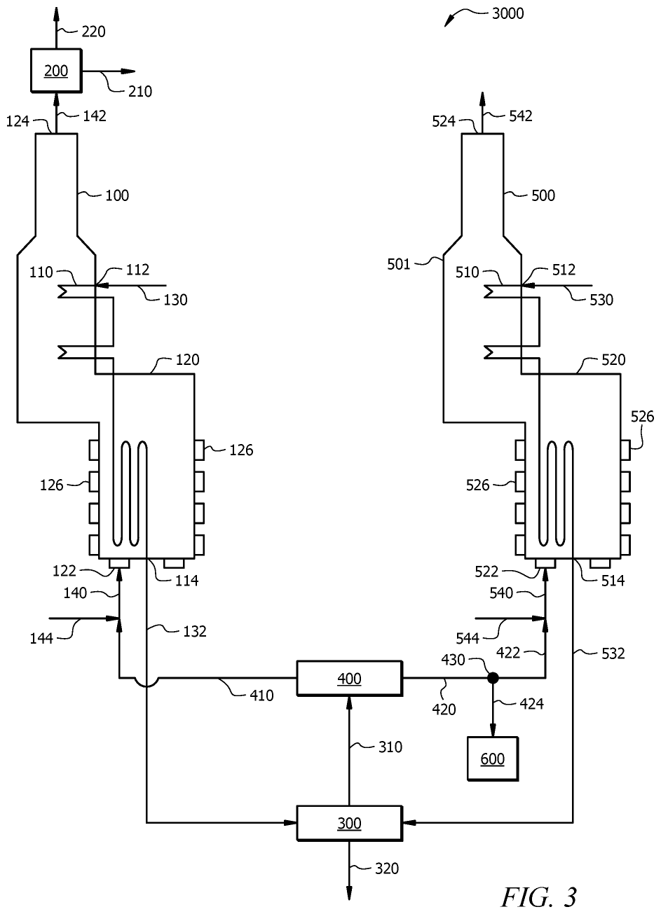

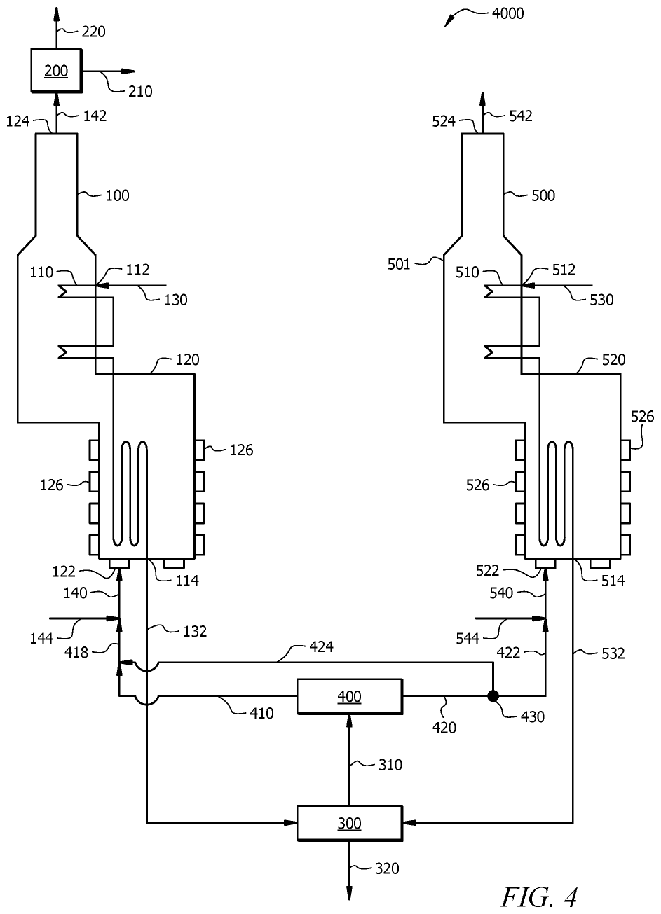

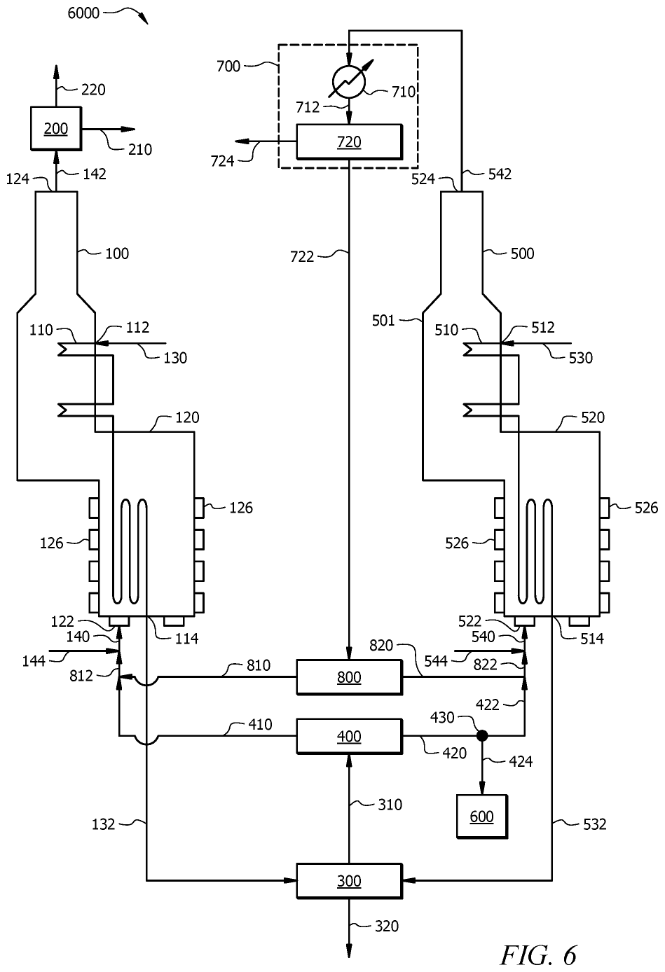

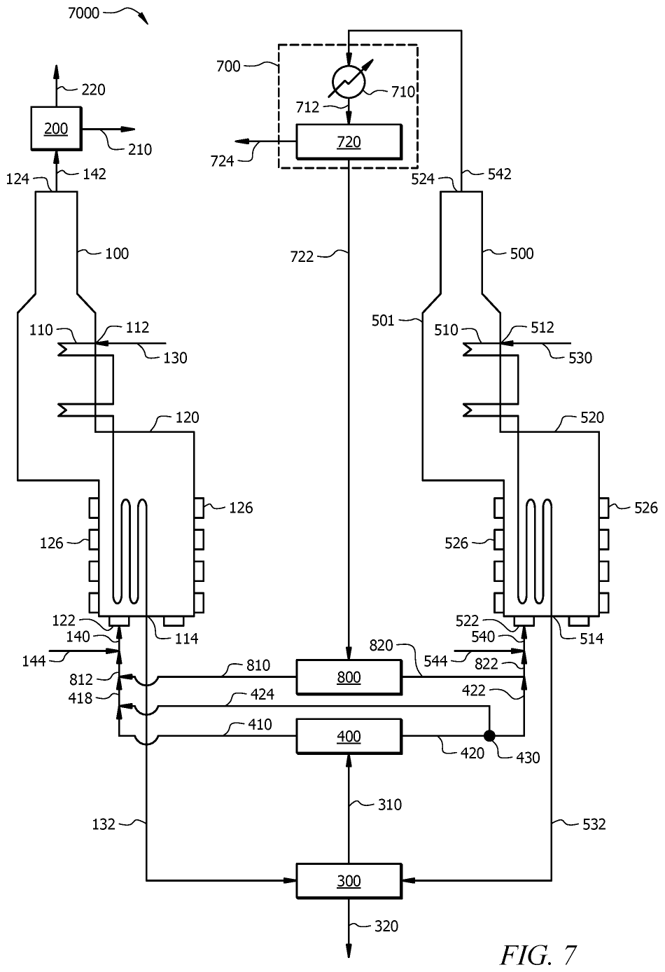

The hydrocarbon cracking systems 3000, 4000, 6000, and 7000 illustrate splitting or selectively directing portions of the hydrogen product stream to different locations in a plant. The hydrocarbon cracking systems 3000 and 6000 in FIGS. 3 and 6 illustrate that the hydrogen product stream can be split or selectively directed to the hydrogen fired furnace and/or to another unit that consumes or stores hydrogen. The hydrocarbon cracking systems 4000 and 7000 in FIGS. 4 and 7 illustrate that the hydrogen product stream can be split or selectively directed to the hydrogen fired furnace and/or to the methane fuel fired furnace (e.g., for scenarios where there is excess hydrogen flow or where the hydrogen fired furnace is offline).

The hydrocarbon cracking systems 5000, 6000, and 7000 in FIGS. 5, 6, and 7 illustrate the recovery of liquid water from the flue gas (referred to herein as the hydrogen combustion product stream) of the hydrogen fired furnace, and electrolysis of the liquid water to produce hydrogen that can be fed or introduced to the hydrogen fired furnace and to produce oxygen that can be fed or introduced to the methane fuel fired furnace for oxygen enriched firing. The use of oxygen from an electrolyzer for oxygen enriched firing of the methane fuel fired furnace(s) produces a flue gas having a higher concentration of carbon dioxide and a lower total volume of flue gas, compared to conventional firing. The lower total volume of flue gas that has higher concentration of carbon dioxide can allow for more efficient carbon capture processing, and smaller carbon capture units. Moreover, the oxygen enriched firing has advantages over oxy-combustion configurations (using pure oxygen) in that, 1) the methane fuel fired furnace(s) can be operated under slight vacuum for oxygen enriched firing whereas oxy-combustion tolerates no amount of nitrogen and the furnaces have to be operated at positive pressure, which can lead to leakage of combustion gases from the furnaces(s), and 2) obtaining pure oxygen for oxy-combustion may require expensive techniques such as cryogenic distillation.

While the hydrocarbon cracking systems 1000, 2000, 3000, 4000, 5000, 6000, and 7000 are described with reference to the equipment illustrated in the figures, it should be appreciated that the action and functionality performed with the equipment can be performed in one or more aspects of the disclosed processes. Like parts of the hydrocarbon cracking systems 1000, 2000, 3000, 4000, 5000, 6000, and 7000 are labeled with the same references and descriptions that apply from one system to another are not reproduced for each system in order to keep the description as concise as possible.

In aspects, each of furnaces in the hydrocarbon cracking systems 1000, 2000, 3000, 4000, 5000, 6000, and 7000 is an olefin cracker, and in particular, an ethylene cracker, configured to produce ethylene from a hydrocarbon feed stream containing ethane. The fuel gas streams that feed to the olefin cracker supply the fuel (e.g., hydrogen for the hydrogen fired furnace and methane for the methane fuel fired furnace) that combusts in the cracker—the heat of combustion providing heat to the conduits inside the crack in which the alkane (e.g., ethane) is flowing and which causes the formation of the olefin (e.g., ethylene) from the alkane (e.g., ethane).

FIG. 1 is a schematic diagram of an embodiment of a hydrocarbon cracking system 1000. The system 1000 includes a methane fuel fired furnace 100, a carbon capture process 200, a first separator 300, a second separator 400, and unit 500.

The methane fuel fired furnace 100 includes conduit 110 that passes hydrocarbon feed components through the interior of the methane fuel fired furnace 100 at temperature sufficient to convert an alkane to an olefin (e.g., ethane to ethylene, propane to propylene, or both). The conduit 110 can be configured to pass through a furnace housing 120 of the methane fuel fired furnace 100 where a fuel gas is combusted in the presence of oxygen to produce heat for cracking of the hydrocarbons in the conduit 110. The methane fuel fired furnace 100 has a first inlet 112 for the conduit 110 that is connected to a hydrocarbon feed stream 130. The methane fuel fired furnace 100 has a first outlet 114 for the conduit 110 that is connected to a hydrocarbon product stream 132. The methane fuel fired furnace 100 has a fuel inlet 122 fluidly coupled to a fuel gas stream 140. The methane fuel fired furnace 100 has a flue gas outlet 124 fluidly coupled to a flue gas stream 142. The furnace housing 120 of the methane fuel fired furnace 100 has burners 126 configured to provide flames for combustion of fuel gas that is received from the fuel gas stream 140. In aspects, the burners 126 are configured with metallurgy for hydrocarbon-based combustion in the furnace housing 120. The fuel gas stream 140 contains air supplied from air stream 144 and methane supplied from methane fuel stream 410 (described in more detail herein).

The hydrocarbon feed stream 130 can include any hydrocarbon or combination of hydrocarbons. For example, the hydrocarbon feed stream 130 can include ethane, propane, butane, or combinations thereof. The hydrocarbon(s) can be received from a hydrocarbon source, such as a pipeline or storage tank.

The hydrocarbon product stream 132 can include methane, hydrogen, cracking products (e.g., ethylene, propylene, butadiene, benzene, or combinations thereof), and uncracked feed hydrocarbons (e.g., ethane, propane, butane, or combinations thereof). In aspects, the hydrocarbon product stream 132 can include methane, hydrogen, uncracked feed hydrocarbon(s), and cracking products.

The fuel gas stream 140 can include gaseous components resulting from the combination of air stream 144 and methane fuel stream 410. For example, the fuel gas stream 140 can include oxygen, nitrogen, and methane. In aspects, the fuel gas stream 140 can additionally contain small amounts of hydrogen as described for the methane fuel stream 410 herein. Generally, the concentration of methane in the fuel gas stream 140 is greater than the concentration of hydrogen in the fuel gas stream 140. For example, a volume ratio of methane:hydrogen in the fuel gas stream 140 can range from about 1.5:1 to about 1,000:1. In aspects, methane from a gas pipeline can be added to the methane fuel stream 410 or to the fuel gas stream 140, to supplement the amount of methane flowing to the methane fuel fired furnace 100.

The flue gas stream 142 can include carbon dioxide, nitrogen, water vapor, uncombusted oxygen, or combinations thereof. “Uncombusted oxygen” refers to oxygen that is fed in fuel gas stream 140 to the methane fuel fired furnace 100 and passes unreacted to the flue gas in flue gas stream 142. In aspects, the flue gas stream 142 can include primarily carbon dioxide, nitrogen, and water vapor; alternatively, greater than 50, 60, 70, 80, or 90 vol % carbon dioxide, nitrogen, and water vapor, based on a total volume of the flue gas stream 142. In a comparative scenario, firing of the hydrogen-rich hydrocarbon fuel stream 310 when fed directly to a furnace can produce a flue gas having carbon dioxide in an amount of 3 vol % or less based on total volume of the flue gas. The flue gas stream 142 can have carbon dioxide present in an amount ranging from about 10 vol % to about 30 vol % based on a total volume of the flue gas stream 142.

The hydrocarbon cracking system 1000 can additionally include a carbon capture process 200, and the flue gas stream 142 of the methane fuel fired furnace 100 can be fluidly connected to the carbon capture process 200. The carbon capture process 200 can be configured to produce a CO2 product stream 210 containing carbon dioxide and residual gas stream 220 containing nitrogen and uncombusted oxygen (“uncombusted oxygen” refers to oxygen that is fed in fuel gas stream 140 to the methane fuel fired furnace 100 and passes unreacted to the flue gas in flue gas stream 142). In aspects, the carbon capture process is not configured to couple with or to receive the hydrogen combustion product stream 542.

The carbon capture process 200 can include equipment for the removal of carbon dioxide from the flue gas stream 142. For example, the carbon capture process 200 can include equipment for absorption, adsorption, membrane separation, carbon dioxide condensation, or a combination thereof, of carbon dioxide from the flue gas received from the flue gas stream 142. In aspects, carbon dioxide can be captured by passing the flue gas stream 142 to an absorber having an amine-based (e.g., monoethanolamine) acid gas sorbent therein (e.g., connected an absorbent regeneration loop). The sorbent absorbs the carbon dioxide while other components of the flue gas (e.g., water vapor, nitrogen, and uncombusted oxygen) bubble through the sorbent to a gas outlet of the absorber. Carbon dioxide can be recovered from the sorbent in a sorbent regenerator, and regenerated sorbent can be recirculated back to the absorber. The recovered carbon dioxide can flow from the sorbent regenerator in the CO2 product stream 210.

In aspects, the CO2 product stream 210 can include carbon dioxide in a range of from about 95 vol % to about 100 vol %; alternatively, from about 95 vol % to about 99 vol %; alternatively, greater than 95, 96, 97, 98, or 99 vol %, based on a total volume of the CO2 product stream 210. In additional aspects, the CO2 product stream 210 can be sent to sequestration or to another process (e.g., for use as inert gas, reactant, coolant in a CO2 cooling loop, or combinations thereof).

The residual gas stream 220 can include nitrogen and uncombusted oxygen. In aspects, the residual gas stream 220 can be free of carbon dioxide. In aspects, the residual gas stream 220 can include carbon dioxide in a range of from 0 vol % to about 5 vol %; alternatively, less than 5, 4, 3, 2, or 1 vol %, based on a total volume of the residual gas stream 220.

The hydrocarbon cracking system 1000 can additionally include a separator 300. The separator 300 is fluidly connected to and configured to receive the hydrocarbon product stream 132. The separator 300 is configured to separate stream 132 into a hydrogen-rich hydrocarbon fuel stream 310 and another hydrocarbon product stream 320. The separator 300 can be embodied as a distillation column in a demethanizer system, configured to remove methane and lighter molecules (e.g., hydrogen) from ethylene and heavier molecules. The hydrocarbon cracking system 1000 can include other equipment to facilitate operation of the demethanizer system, such as a quench tower, a caustic tower, dryers, compression stage, a deethanizer system, a cold box, heat exchangers, or combinations thereof.

The hydrogen-rich hydrocarbon fuel stream 310 can include hydrogen, methane, and other hydrocarbons (e.g., uncracked feed components such as ethane, propane, butane, or combinations thereof; cracking products such as ethylene, propylene, butene, butadiene, benzene, or combinations thereof). In aspects, the hydrogen-rich hydrocarbon fuel stream 310 can include hydrogen in a range of from about 40 vol % to about 100 vol %; alternatively, in a range of from about 40 vol % to about 90 vol %; alternatively, in a range of from about 90 vol % to about 100 vol % based on a total volume of the hydrogen-rich hydrocarbon fuel stream 310. In additional aspects, the hydrogen-rich hydrocarbon fuel stream 310 can include methane in a range of from about 10 vol % to about 85 vol % based on a total volume of the hydrogen-rich hydrocarbon fuel stream 310.

The hydrocarbon product stream 320 can include cracking products (e.g., ethylene, propylene, butadiene, benzene, or combinations thereof), uncracked feed hydrocarbons (e.g., ethane, propane, butane, or combinations thereof), or combinations thereof, that are separated in the separator 300.

The hydrocarbon cracking system 1000 can additionally include a separator 400. The separator 400 is fluidly connected to and configured to receive the hydrogen-rich hydrocarbon fuel stream 310. The separator 400 is configured to separate the hydrogen-rich hydrocarbon fuel stream 310 into the methane fuel stream 410 containing methane and a hydrogen product stream 420 containing hydrogen. The separator 400 can be embodied as vessels and equipment configured to separate methane from hydrogen by pressure swing absorption, membrane separation, cryogenic separations (e.g., cryogenic distillation columns), or combinations thereof.

The methane fuel stream 410 can be fluidly connected to the fuel gas stream 140 such that the methane in methane fuel stream 410 feeds to the methane fuel fired furnace 100 via the fuel gas stream 140 (along with air received via air stream 144). The methane fuel stream 410 can include methane in a range of from about 60 vol % to about 100 vol % based on a total volume of the methane fuel stream 410. In aspects, the methane fuel stream 410 can include hydrogen in a range of from 0 vol % to about 40 vol % based on a total volume of the methane fuel stream 410.

The hydrogen product stream 420 can include hydrogen in a range of from about 88 vol % to about 99.9 vol %; alternatively, from about 90 vol % to about 99.9 vol %; alternatively, from about 95 vol % to about 99.9 vol %, based on a total volume of the hydrogen product stream 420. In aspects, the hydrogen product stream 420 is not pure hydrogen, e.g., does not contain 100 vol % hydrogen. In these aspects, the hydrogen product stream 420 can contain methane in a range of from about 0.1 vol % to about 12 vol %; alternatively, from about 0.1 vol % to about 10 vol %; alternatively, from about 0.1 to about 5 vol %, based on a total volume of the hydrogen product stream 420.

A unit 500 can be configured to fluidly couple with and to receive the hydrogen product stream 420. The unit 500 can include i) a hydrogen fired furnace, ii) a hydrogen fired boiler, iii) a hydrogen sales gas pipeline, iv) a storage container, v) another process, or vi) combinations thereof.

In aspects, the hydrocarbon cracking system 1000 can include additional methane fuel fired furnaces, each configured to receive the hydrocarbon feed stream 130 and fuel gas stream 140, each configured to produce flue gas stream 142 and hydrocarbon product stream 132, where each additional flue gas stream 142 additionally fluidly couples to the carbon capture process 200, and where each additional hydrocarbon product stream 132 additionally fluidly couples to the separator 300.

A method performed with system 1000 can include separating the hydrogen-rich hydrocarbon fuel stream 310 into the methane fuel stream 410 and the hydrogen product stream 420, combusting the methane fuel stream 410 in the methane fuel fired furnace 100 to produce the flue gas stream 142, and feeding the flue gas stream 142 to the carbon capture process 200. The method performed with system 1000 can also include separating the flue gas stream 142 into the CO2 product stream 210 and the residual gas stream 220.

The method performed with system 1000 can also include cracking, in the methane fuel fired furnace 100, the hydrocarbon feed stream 130 to produce the hydrocarbon product stream 132 by combusting the methane fuel stream 410; receiving the hydrocarbon product stream 132 into the separator 300; and separating the hydrocarbon product stream 132 in the separator 300 to produce the hydrogen-rich hydrocarbon fuel stream 310 and the hydrocarbon product stream 320.

The method performed with system 1000 can also include feeding the hydrogen product stream 420 to a unit 500 that can be embodied as i) a hydrogen fired furnace, ii) a hydrogen fired boiler, iii) a hydrogen sales gas pipeline, iv) a storage container, v) another process, or vi) combinations thereof.

The streams in the method performed with system 1000 can have any composition described herein. For example, the hydrogen-rich hydrocarbon fuel stream 310 can include hydrogen, methane, and other light hydrocarbons. Hydrogen can be present in the hydrogen-rich hydrocarbon fuel stream 310 in a range of from about 40 vol % to about 100 vol %; alternatively, in a range of from about 40 vol % to about 90 vol %; alternatively, in a range of from about 90 vol % to about 100 vol % based on a total volume of the hydrogen-rich hydrocarbon fuel stream 310. Additionally, the flue gas stream 142 can include carbon dioxide, nitrogen, and water vapor. In aspects of the method performed with system 1000, the hydrogen product stream 420 is not fed to the methane fuel fired furnace 100. In additional or alternative aspects of the method performed with system 1000, the hydrogen product stream 420 is not connected, and does not flow to, the carbon capture process 200.

FIG. 2 is a schematic diagram of an embodiment of another embodiment of a hydrocarbon cracking system 2000. The system 2000 includes the methane fuel fired furnace 100, the carbon capture process 200, the first separator 300, the second separator 400, and the unit 500 embodied as a hydrogen fired furnace 501.

The arrangement of the methane fuel fired furnace 100, the carbon capture process 200, the first separator 300, and the second separator 400 in system 2000 is the same as described for system 1000, with additional aspects and features as described below.

The hydrocarbon cracking system 2000 can include the hydrogen fired furnace 501. The hydrogen fired furnace 501 includes conduit 510 that passes hydrocarbon feed components through the interior of the hydrogen fired furnace 501 at temperature sufficient to convert an alkane to an olefin (e.g., ethane to ethylene, propane to propylene, or both). The conduit 510 can be configured to pass through a furnace housing 520 of the hydrogen fired furnace 501 where a fuel gas is combusted in the presence of oxygen to produce heat for cracking the hydrocarbons in the conduit 510. The hydrogen fired furnace 501 has a first inlet 512 for the conduit 510 that is connected to a hydrocarbon feed stream 530. The hydrogen fired furnace 501 has a first outlet 514 for the conduit 510 that is connected to a hydrocarbon product stream 532. The hydrogen fired furnace 501 has a fuel inlet 522 fluidly coupled to a fuel gas stream 540. The hydrogen fired furnace 501 has a flue gas outlet 524 fluidly coupled to a hydrogen combustion product stream 542. The hydrogen combustion product stream 542 is the flue gas emitted from the hydrogen fired furnace 501 and is referred to herein as the hydrogen combustion product stream 542. The furnace housing 520 of the hydrogen fired furnace 501 has burners 526 configured to provide flames for combustion of fuel gas that is received from the fuel gas stream 540. In aspects, the burners 526 are configured with metallurgy for hydrogen-based combustion in the furnace housing 520.

The fuel gas stream 540 can include gaseous components resulting from the combination of air stream 544 and hydrogen product stream 420. That is, the fuel gas stream 540 can contain air supplied from air stream 544 and hydrogen supplied from hydrogen product stream 420, which in system 2000 can be referred to as a hydrogen fuel stream. In aspects, the air stream 144 and air stream 544 can contain air supplied from the same air source.

In the system 2000, the separator 300 is fluidly connected to and configured to receive both the hydrocarbon product stream 532 and the hydrocarbon product stream 132. The separator 300 is configured to separate streams 532 and 132 into the hydrogen-rich hydrocarbon fuel stream 310 and the hydrocarbon product stream 320.

The hydrocarbon feed stream 530 can include any hydrocarbon or combination of hydrocarbons. For example, the hydrocarbon feed stream 530 can include ethane, propane, butane, or combinations thereof. In aspects, the hydrocarbon feed stream 530 can contain the same composition of hydrocarbons as the hydrocarbon feed stream 130. Stated differently, the hydrocarbon feed stream 530 and the hydrocarbon feed stream 130 can receive feed hydrocarbons from the same hydrocarbon source.

The hydrocarbon product stream 532 can include methane, hydrogen, cracking products (e.g., ethylene, propylene, butadiene, benzene, or combinations thereof), uncracked feed hydrocarbons (e.g., ethane, propane, butane, or combinations thereof), or combinations thereof. For example, the hydrocarbon product stream 532 can include methane, hydrogen, uncracked feed hydrocarbon(s) (e.g., ethane, propane, butane, or combinations thereof), and cracking products (e.g., ethylene, propylene, butadiene, benzene, or combinations thereof).

The hydrogen product stream 420 can be fluidly connected to the fuel gas stream 540 such that the hydrogen in hydrogen product stream 420 feeds to the hydrogen fired furnace 501 via the fuel gas stream 540 (along with air received via air stream 544). The fuel gas stream 540 can include gaseous components resulting from the combination of air stream 144 and hydrogen product stream 420. For example, the fuel gas stream 540 can include oxygen, nitrogen, and hydrogen. In aspects, the fuel gas stream 540 can additionally contain small amounts of methane as described for the hydrogen product stream 420 herein. The concentration of hydrogen in the fuel gas stream 540 is greater than the concentration of methane in the fuel gas stream 540. For example, a volume ratio of hydrogen:methane in the fuel gas stream 540 can range from about 7:1 to about 1,000:1.

The hydrogen combustion product stream 542 can include water vapor, nitrogen, and uncombusted oxygen. “Uncombusted oxygen” refers to oxygen that is fed in fuel gas stream 540 to the hydrogen fired furnace 501 and passes unreacted to the flue gas in hydrogen combustion product stream 542. In aspects, the hydrogen combustion product stream 542 is free of carbon dioxide. “Free of carbon dioxide” can include less than 1, 0.9, 0.8, 0.7, 0.6, 0.5, 0.4, 0.3, 0.2, or 0.1 vol % carbon dioxide based on a total volume of the hydrogen combustion product stream 542. In further aspects, the hydrogen combustion product stream 542 is not fed to the carbon capture process 200.

The separator 300 can be fluidly connected to and configured to receive both the hydrocarbon product stream 132 and the hydrocarbon product stream 532. The separator 300 in system 2000 is configured to separate the hydrocarbon product stream 132 and the hydrocarbon product stream 532 into the hydrogen-rich hydrocarbon fuel stream 310 and the hydrocarbon product stream 320.

In aspects, the hydrocarbon cracking system 2000 can include additional methane fuel fired furnaces, each configured to receive the hydrocarbon feed stream 130 and fuel gas stream 140, each configured to produce flue gas stream 142 and hydrocarbon product stream 132, where each additional flue gas stream 142 additionally fluidly couples to the carbon capture process 200, and where each additional hydrocarbon product stream 132 additionally fluidly couples to the separator 300.

In aspects, the hydrocarbon cracking system 2000 can include additional hydrogen fired furnaces, each configured to receive the hydrocarbon feed stream 530 and fuel gas stream 540, each configured to produce hydrogen combustion product stream 542 and hydrocarbon product stream 532, where each additional hydrogen combustion product stream 542 additionally fluidly couples to the carbon capture process 200, and where each additional hydrocarbon product stream 532 additionally fluidly couples to the separator 300.

A method performed with system 2000 can include separating the hydrogen-rich hydrocarbon fuel stream 310 into the methane fuel stream 410 and the hydrogen product stream 420, combusting the methane fuel stream 410 in the methane fuel fired furnace 100 to produce the flue gas stream 142, combusting the hydrogen product stream 420 in the hydrogen fired furnace 501 to produce the hydrogen combustion product stream 542, and feeding the flue gas stream 142 to the carbon capture process 200.

The method performed with system 2000 can also include separating the flue gas stream 142 into the CO2 product stream 210 and the residual gas stream 220.

The method performed with system 2000 can also include cracking, in the methane fuel fired furnace 100, the hydrocarbon feed stream 130 to produce the hydrocarbon product stream 132 by combusting the methane fuel stream 410; and cracking, in the hydrogen fired furnace 501, the hydrocarbon feed stream 530 to produce the hydrocarbon product stream 532 by combusting the hydrogen product stream 420.

The method performed with system 2000 can also include receiving the hydrocarbon product stream 132 and the hydrocarbon product stream 532 into the separator 300, and separating the hydrocarbon product stream 132 and the hydrocarbon product stream 532 in the separator 300 to produce the hydrogen-rich hydrocarbon fuel stream 310 and the hydrocarbon product stream 320.

The method performed with system 2000 can also include feeding the hydrogen product stream 420 to the hydrogen fired furnace 501.

The streams in the method performed with system 2000 can have any composition described herein. For example, the hydrogen-rich hydrocarbon fuel stream 310 can include hydrogen, methane, and other light hydrocarbons. Hydrogen can be present in the hydrogen-rich hydrocarbon fuel stream 310 in a range of from about 40 vol % to about 100 vol %; alternatively, in a range of from about 40 vol % to about 90 vol %; alternatively, in a range of from about 90 vol % to about 100 vol % based on a total volume of the hydrogen-rich hydrocarbon fuel stream 310. Additionally, the flue gas stream 142 can include carbon dioxide, nitrogen, and water vapor. Additionally, the hydrogen combustion product stream 542 can include nitrogen, uncombusted oxygen, and water vapor, and can be free of carbon dioxide. In aspects of the method performed with system 2000, the hydrogen product stream 420 is not feed to the methane fuel fired furnace 100. In additional or alternative aspects of the method performed with system 2000, the hydrogen product stream 420 is not connected to, and does not flow to, the carbon capture process 200.

FIG. 3 is a schematic diagram of an alternative embodiment of a hydrocarbon cracking system 3000. The system 3000 in FIG. 3 includes equipment described for the system 2000 in FIG. 2, including the methane fuel fired furnace 100, the carbon capture process 200, the separator 300, the separator 400, and the hydrogen fired furnace 501. The system 3000 is similar to system 2000 in FIG. 2, with the addition of a splitter 430 that is fluidly connected to the hydrogen product stream 420 and that is configured to split or selectively direct the hydrogen product stream 420 into a first stream 422, a second stream 424, or both the first stream 422 and the second stream 424. The first stream 422 fluidly connects to the fuel gas stream 540. The hydrocarbon cracking system 3000 also includes a unit 600, and the second stream 424 is fluidly connected to the unit 600.

The splitter 430 can be embodied as any conduit connector, conduit, instrumentation, valving, or combinations thereof that are configured to split or selectively direct the hydrogen product stream 420 into the first stream 422, the second stream 424, or into both the first stream 422 and the second stream 424. In some aspect, the splitter 430 can continuously split flow of stream 420 into both the first stream 422 and the second stream 424, and a split volume ratio can be in a range of 100:1 to 1:100. Alternatively, the splitter 430 can selectively direct flow to the first stream 422 or to the second stream 424 but not both. Alternatively, the splitter 430 can selectively direct flow of hydrogen product from flowing only to hydrogen fired furnace 501 via first stream 422 to only to unit 600 via second stream 424; alternatively, from flowing only to unit 600 via second stream 424 to only to hydrogen fired furnace 501 via first stream 422; alternatively, from flowing to both hydrogen fired furnace 501 and to unit 600 to flowing only to the hydrogen fired furnace 501; alternatively, from flowing to both hydrogen fired furnace 501 and to unit 600 to flowing only to the unit 600. Selectively directing flow can include closing one or more valves to stop flow in a given stream and opening one or more valves to start flow in another given stream (e.g., close a valve in stream 422, open a valve in stream 424).

In aspects, the first stream 422 and the second stream 424 can have the same composition as the hydrogen product stream 420 (described herein).

The fuel gas stream 540 can include gaseous components resulting from the combination of air stream 544 and the first stream 422.

Unit 600 can include i) a hydrogen fired boiler, ii) a hydrogen sales gas pipeline, iii) a storage container, iv) another process, or v) combinations thereof.

In aspects, the hydrocarbon cracking system 3000 can include additional methane fuel fired furnaces, each configured to receive the hydrocarbon feed stream 130 and fuel gas stream 140, each configured to produce flue gas stream 142 and hydrocarbon product stream 132, where each additional flue gas stream 142 additionally fluidly couples to the carbon capture process 200, and where each additional hydrocarbon product stream 132 additionally fluidly couples to the separator 300.

In aspects, the hydrocarbon cracking system 3000 can include additional hydrogen fired furnaces, each configured to receive the hydrocarbon feed stream 530 and fuel gas stream 540, each configured to produce hydrogen combustion product stream 542 and hydrocarbon product stream 532, where each additional hydrogen combustion product stream 542 additionally fluidly couples to the carbon capture process 200, and where each additional hydrocarbon product stream 532 additionally fluidly couples to the separator 300.

A method performed with system 3000 can include separating the hydrogen-rich hydrocarbon fuel stream 310 into the methane fuel stream 410 and the hydrogen product stream 420, combusting the methane fuel stream 410 in the methane fuel fired furnace 100 to produce the flue gas stream 142, splitting the hydrogen product stream 420 into a first stream 422 and a second stream 424, feeding the first stream 422 to the hydrogen fired furnace 501, and combusting the first stream 422 of the hydrogen product stream 420 in the hydrogen fired furnace 501 to produce the hydrogen combustion product stream 542.

The method performed with system 3000 can also include feeding the flue gas stream 142 to the carbon capture process 200, and feeding the second stream 424 to i) another hydrogen fired furnace, ii) a hydrogen fired boiler, iii) a sales gas pipeline, iv) a storage container, v) another process, or vi) a combination thereof.

The method performed with system 3000 can also include separating the flue gas stream 142 into the CO2 product stream 210 and the residual gas stream 220.

The method performed with system 3000 can also include cracking, in the methane fuel fired furnace 100, the hydrocarbon feed stream 130 to produce the hydrocarbon product stream 132 by combusting the methane fuel stream 410; and cracking, in the hydrogen fired furnace 501, the hydrocarbon feed stream 530 to produce the hydrocarbon product stream 532 by combusting the first stream 422 of the hydrogen product stream 420.

The method performed with system 3000 can also include receiving the hydrocarbon product stream 132 and the hydrocarbon product stream 532 into the separator 300, and separating the hydrocarbon product stream 132 and the hydrocarbon product stream 532 in the separator 300 to produce the hydrogen-rich hydrocarbon fuel stream 310 and the hydrocarbon product stream 320.

The method performed with system 3000 can also include feeding the first stream 422 of the hydrogen product stream 420 to the hydrogen fired furnace 501 and feeding the second stream 424 of the hydrogen product stream 420 to the unit 600.

Another method performed with system 3000 can include separating the hydrogen-rich hydrocarbon fuel stream 310 into the methane fuel stream 410 and the hydrogen product stream 420, combusting the methane fuel stream 410 in the methane fuel fired furnace 100 to produce the flue gas stream 142, selectively directing the hydrogen product stream 420 into a first stream 422, feeding the first stream 422 to the hydrogen fired furnace 501, and combusting the first stream 422 of the hydrogen product stream 420 in the hydrogen fired furnace 501 to produce the hydrogen combustion product stream 542. This alternative method performed with system 3000 can also include switching or selectively directing (e.g., by splitter 430) a flow of the hydrogen product stream 420: 1) from flowing only to the hydrogen fired furnace 501 via stream 422 to only to the unit 600 via stream 424, 2) from flowing only to the hydrogen fired furnace 501 via stream 422 to flow to both the furnace 501 via stream 422 and to the unit 600 via stream 424. In aspects where the method includes selectively directing hydrogen product via second stream 424 only to the unit 600, the method can additionally include 1) from flowing only to the unit 600 via stream 424 to only to the hydrogen fired furnace 501 via stream 422, or 2) from flowing only to the unit 600 via stream 424 to flow to both the furnace 501 via stream 422 and to the unit 600 via stream 424. Selectively directing can include actuating a valve from a closed position to an open position and actuating another valve from an open position to a closed position.

The alternative method performed with system 3000 can also include feeding the flue gas stream 142 to the carbon capture process 200, and when hydrogen product flows in second stream 424, feeding the second stream 424 to i) another hydrogen fired furnace, ii) a hydrogen fired boiler, iii) a sales gas pipeline, iv) a storage container, v) another process, or vi) a combination thereof.

The alternative method performed with system 3000 can also include separating the flue gas stream 142 into the CO2 product stream 210 and the residual gas stream 220.

The alternative method performed with system 3000 can also include cracking, in the methane fuel fired furnace 100, the hydrocarbon feed stream 130 to produce the hydrocarbon product stream 132 by combusting the methane fuel stream 410; and cracking, in the hydrogen fired furnace 501, the hydrocarbon feed stream 530 to produce the hydrocarbon product stream 532 by combusting the first stream 422 of the hydrogen product stream 420 (when hydrogen product flows via first stream 422).

The alternative method performed with system 3000 can also include receiving the hydrocarbon product stream 132 and the hydrocarbon product stream 532 into the separator 300, and separating the hydrocarbon product stream 132 and the hydrocarbon product stream 532 in the separator 300 to produce the hydrogen-rich hydrocarbon fuel stream 310 and the hydrocarbon product stream 320.

The streams in the methods performed with system 3000 can have any composition described herein. For example, the hydrogen-rich hydrocarbon fuel stream 310 can include hydrogen, methane, and other light hydrocarbons. Hydrogen can be present in the hydrogen-rich hydrocarbon fuel stream 310 in a range of from about 40 vol % to about 100 vol %; alternatively, in a range of from about 40 vol % to about 90 vol %; alternatively, in a range of from about 90 vol % to about 100 vol % based on a total volume of the hydrogen-rich hydrocarbon fuel stream 310. Additionally, the flue gas stream 142 can include carbon dioxide, nitrogen, and water vapor. Additionally, the hydrogen combustion product stream 542 can include nitrogen, uncombusted oxygen, and water vapor, and can be free of carbon dioxide. In aspects of the method performed with system 3000, none of the hydrogen product stream 420, the first stream 422 of the hydrogen product stream 420, and the second stream 424 of the hydrogen product stream 420 is fed to the methane fuel fired furnace 100. In additional or alternative aspects of the method performed with system 3000, none of the hydrogen product stream 420, the first stream 422 of the hydrogen product stream 420, and the second stream 424 of the hydrogen product stream 420 is connected to, and none flow to, the carbon capture process 200.

FIG. 4 is a schematic diagram of an alternative embodiment of a hydrocarbon cracking system 4000. The system 4000 in FIG. 4 includes equipment described for the system 3000 in FIG. 3, including the methane fuel fired methane fuel fired furnace 100, the carbon capture process 200, the separator 300, the separator 400, the hydrogen fired furnace 501, and the splitter 430.

The fuel gas stream 140 can include gaseous components resulting from the combination of air stream 144, methane fuel stream 410, and second stream 424. FIG. 4 illustrates that the second stream 424 of the hydrogen product stream 420 is fluidly connected to the fuel gas stream 140. More particularly, the second stream 424 of the hydrogen product stream 420 is configured to combine with the methane fuel stream 410 to form a combined stream 418. The combined stream 418 is configured to combine with the air stream 144 to form the fuel gas stream 140. In aspects, the order of combination of streams 410, 424, and 144 can be different than illustrated in FIG. 4. For example, the streams 410, 424, and 144 can combine at a single junction point to form the fuel gas stream 140.

The fuel gas stream 540 can include gaseous components resulting from the combination of air stream 544 and the first stream 422.

The splitter 430 in FIG. 4 can be configured similarly to the splitter 430 in FIG. 3. The splitter 430 can be embodied as any conduit connector, conduit, instrumentation, valving, or combinations thereof that are configured to split or selectively direct the hydrogen product stream 420 into the first stream 422, the second stream 424, or into both the first stream 422 and the second stream 424. In some aspect, the splitter 430 can continuously split flow of stream 420 into both the first stream 422 and the second stream 424, and a split volume ratio can be in a range of 100:1 to 1:100. Alternatively, the splitter 430 can selectively direct flow from flowing only to hydrogen fired furnace 501 via first stream 422 to only to methane fuel fired furnace 100 via second stream 424; alternatively, the splitter 430 can selectively direct flow from flowing only to methane fuel fired furnace 100 via second stream 424 to only to hydrogen fired furnace 501 via first stream 422. Selectively directing flow can include closing one or more valves to stop flow in a given stream and opening one or more valves to start flow in another given stream (e.g., close a valve in stream 422, open a valve in stream 424).

The configuration in FIG. 4 can be useful for directing hydrogen product to the methane fuel fired furnace 100. For example, in a scenario where the flow of hydrogen in the hydrogen product stream 420 is greater than the capacity of the hydrogen fired furnace 501, excess hydrogen product can be routed to the methane fuel fired furnace 100 via second stream 424 and splitter 430 (in addition to flow of hydrogen product continuing in first stream 422). In another example, in a scenario where the hydrogen fired furnace 501 is taken offline, the flow of hydrogen in the hydrogen product stream 420 can be directed to flow only in second stream 424 to the methane fuel fired furnace 100.

In aspects, the hydrocarbon cracking system 4000 can include additional methane fuel fired furnaces, each configured to receive the hydrocarbon feed stream 130 and fuel gas stream 140, each configured to produce flue gas stream 142 and hydrocarbon product stream 132, where each additional flue gas stream 142 additionally fluidly couples to the carbon capture process 200, and where each additional hydrocarbon product stream 132 additionally fluidly couples to the separator 300.

In aspects, the hydrocarbon cracking system 4000 can include additional hydrogen fired furnaces, each configured to receive the hydrocarbon feed stream 530 and fuel gas stream 540, each configured to produce hydrogen combustion product stream 542 and hydrocarbon product stream 532, where each additional hydrogen combustion product stream 542 additionally fluidly couples to the carbon capture process 200, and where each additional hydrocarbon product stream 532 additionally fluidly couples to the separator 300.

A method performed with system 4000 can include separating the hydrogen-rich hydrocarbon fuel stream 310 into the methane fuel stream 410 and the hydrogen product stream 420, splitting the hydrogen product stream 420 into a first stream 422 and a second stream 424, feeding the first stream 422 to the hydrogen fired furnace 501, combusting the first stream 422 of the hydrogen product stream 420 in the hydrogen fired furnace 501 to produce the hydrogen combustion product stream 542, feeding the second stream 424 and the methane fuel stream 410 to the methane fuel fired furnace 100, and combusting the second stream 424 and the methane fuel stream 410 in the methane fuel fired furnace 100 to produce the flue gas stream 142. In aspects, before feeding the second stream 424 to the methane fuel fired furnace 100, the method can include combining the second stream 424 with methane fuel stream 410, air stream 144, or both, to form the fuel gas stream 140, and flowing the second stream 424 with methane and air into the furnace 100 via the fuel gas stream 140.

The method performed with system 4000 can also include feeding the flue gas stream 142 to the carbon capture process 200.

The method performed with system 4000 can also include separating the flue gas stream 142 into the CO2 product stream 210 and the residual gas stream 220.

The method performed with system 4000 can also include cracking, in the methane fuel fired furnace 100, the hydrocarbon feed stream 130 to produce the hydrocarbon product stream 132 by combusting the methane fuel stream 410 and the second stream 424 of the hydrogen product stream 420; and cracking, in the hydrogen fired furnace 501, the hydrocarbon feed stream 530 to produce the hydrocarbon product stream 532 by combusting the first stream 422 of the hydrogen product stream 420.

The method performed with system 4000 can also include receiving the hydrocarbon product stream 132 and the hydrocarbon product stream 532 into the separator 300, and separating the hydrocarbon product stream 132 and the hydrocarbon product stream 532 in the separator 300 to produce the hydrogen-rich hydrocarbon fuel stream 310 and the hydrocarbon product stream 320.

The method performed with system 4000 can also include feeding the first stream 422 of the hydrogen product stream 420 to the hydrogen fired furnace 501 and feeding the second stream 424 of the hydrogen product stream 420 to the unit 600.

Another method performed with system 4000 can include separating the hydrogen-rich hydrocarbon fuel stream 310 into the methane fuel stream 410 and the hydrogen product stream 420, combusting the methane fuel stream 410 in the methane fuel fired furnace 100 to produce the flue gas stream 142, selectively directing the hydrogen product stream 420 into a first stream 422, feeding the first stream 422 to the hydrogen fired furnace 501, and combusting the first stream 422 of the hydrogen product stream 420 in the hydrogen fired furnace 501 to produce the hydrogen combustion product stream 542. This alternative method performed with system 4000 can also include switching or selectively directing (e.g., by splitter 430) a flow of the hydrogen product stream 420: 1) from flowing only to the hydrogen fired furnace 501 via stream 422 to only to the methane fuel fired furnace 100 via stream 424, 2) from flowing only to the hydrogen fired furnace 501 via stream 422 to flow to both the furnace 501 via stream 422 and to the methane fuel fired furnace 100 via stream 424. In aspects where the method includes selectively directing hydrogen product via second stream 424 only to the methane fuel fired furnace 100, the method can additionally include 1) from flowing only to the methane fuel fired furnace 100 via stream 424 to only to the hydrogen fired furnace 501 via stream 422, or 2) from flowing only to the methane fuel fired furnace 100 via stream 424 to flow to both the furnace 501 via stream 422 and to the methane fuel fired furnace 100 via stream 424. Selectively directing can include actuating a valve from a closed position to an open position and actuating another valve from an open position to a closed position.

The alternative method performed with system 4000 can also include feeding the flue gas stream 142 to the carbon capture process 200, and when hydrogen product flows in second stream 424, feeding the second stream 424 to i) another hydrogen fired furnace, ii) a hydrogen fired boiler, iii) a sales gas pipeline, iv) a storage container, v) another process, or vi) a combination thereof.

The alternative method performed with system 4000 can also include separating the flue gas stream 142 into the CO2 product stream 210 and the residual gas stream 220.

The alternative method performed with system 4000 can also include cracking, in the methane fuel fired furnace 100, the hydrocarbon feed stream 130 to produce the hydrocarbon product stream 132 by combusting the methane fuel stream 410; and cracking, in the hydrogen fired furnace 501, the hydrocarbon feed stream 530 to produce the hydrocarbon product stream 532 by combusting the first stream 422 of the hydrogen product stream 420 (when hydrogen product flows via first stream 422).

The alternative method performed with system 4000 can also include receiving the hydrocarbon product stream 132 and the hydrocarbon product stream 532 into the separator 300, and separating the hydrocarbon product stream 132 and the hydrocarbon product stream 532 in the separator 300 to produce the hydrogen-rich hydrocarbon fuel stream 310 and the hydrocarbon product stream 320.

The streams in the methods performed with system 4000 can have any composition described herein. For example, the hydrogen-rich hydrocarbon fuel stream 310 can include hydrogen, methane, and other light hydrocarbons. Hydrogen can be present in the hydrogen-rich hydrocarbon fuel stream 310 in a range of from about 40 vol % to about 100 vol %; alternatively, in a range of from about 40 vol % to about 90 vol %; alternatively, in a range of from about 90 vol % to about 100 vol % based on a total volume of the hydrogen-rich hydrocarbon fuel stream 310. Additionally, the flue gas stream 142 can include carbon dioxide, nitrogen, and water vapor. Additionally, the hydrogen combustion product stream 542 can include nitrogen, uncombusted oxygen, and water vapor, and can be free of carbon dioxide. In aspects of the method performed with system 4000, none of the hydrogen product stream 420, the first stream 422 of the hydrogen product stream 420, and the second stream 424 of the hydrogen product stream 420 is fed to the methane fuel fired furnace 100. In additional or alternative aspects of the method performed with system 4000, none of the hydrogen product stream 420, the first stream 422 of the hydrogen product stream 420, and the second stream 424 of the hydrogen product stream 420 is connected to, and none flow to, the carbon capture process 200.

FIGS. 5, 6, and 7 recover water from the hydrogen combustion product stream 542 of a hydrogen fired furnace 501, and utilize electrolysis to recover an O2 product and a hydrogen product from the water. The O2 product can be fed to the methane fuel fired furnace 100 to enrich the fuel gas stream 140 with oxygen. The hydrogen product can flow to various locations as described below, and is a second source of hydrogen for a hydrogen fired furnace 501 or other unit that stores or consumes hydrogen.

FIG. 5 is a schematic diagram of an alternative embodiment of a hydrocarbon cracking system 5000. The system 5000 in FIG. 5 includes the configuration of equipment as described for the system 2000 in FIG. 2, and additionally includes a separator 700 and electrolyzer 800.

The separator 700 is fluidly connected to and configured to receive the hydrogen combustion product stream 542. The separator 700 can include a heat exchanger 710, which can also be referred to as a condenser, and a vapor/liquid separator 720. The heat exchanger 710 can have an inlet fluidly coupled to the hydrogen combustion product stream 542. The heat exchanger 710 is configured to cool the water vapor, nitrogen, and uncombusted oxygen in the hydrogen combustion product stream 542 using a cooling medium in heat exchange contact (e.g., via shell and tube or plate and frame heat exchange configuration) with the components of the hydrogen combustion product stream 542. The water vapor, nitrogen, and uncombusted oxygen in the hydrogen combustion product stream 542 are cooled to a temperature sufficient to condense the water vapor in hydrogen combustion product stream 542 to liquid phase. Cooled stream 712 is fluidly coupled to an outlet of the heat exchanger 710 and to the vapor/liquid separator 720. The cooled stream 712 can be a two-phase stream containing water in liquid phase and nitrogen and uncombusted oxygen in gas phase. The vapor/liquid separator 720 is configured to fluidly couple to and to receive the cooled stream 712 and to separate the liquid phase from the gas phase to produce water stream 722 containing water and the residual gas stream 724 containing nitrogen and uncombusted oxygen.

The water stream 722 can include water in a range of greater than 95, 96, 97, 98, or 99 wt % based on a total weight of the water stream 722. In aspects, the water stream 722 is free of carbon dioxide.

The residual gas stream 724 can include nitrogen and uncombusted oxygen. In aspects, the residual gas stream 724 can be free of carbon dioxide. In aspects, the residual gas stream 724 can include carbon dioxide in a range of from 0 vol % to about 5 vol %; alternatively, less than 5, 4, 3, 2, or 1 vol %, based on a total volume of the residual gas stream 724.

The electrolyzer 800 is configured to fluidly couple to and to receive the water stream 722. The electrolyzer 800 also can be embodied as one or more electrolysis units. The electrolyzer 800 can have a housing that contains a cell having a cathode side, an anode side, and a membrane or solid ceramic material positioned between the cathode side and the anode side. The electrolyzer 800 can be embodied as a solid oxide electrolyzer, an alkaline electrolyzer, or a proton exchange membrane electrolyzer. An example of a commercially available electrolyzer is the Cummins HYLYZER® proton exchange membrane electrolyzer.

The electrolyzer 800 can be configured to separate the hydrogen atoms from the oxygen atom in a water molecule such that oxygen molecules (O2) flow in O2 product stream 810 and hydrogen molecules (H2) flow in hydrogen product stream 820.

Water electrolysis is an electrochemical reaction which takes place when electricity is applied to the anode side and the cathode side of the electrolyzer 800, which causes the water to split into its component molecules, hydrogen (H2) and oxygen (O2). The electrolyzer 800 can be powered by electricity from any power generation source, including renewable energy sources, such as solar, hydroelectric, or wind energy. The capacity of the electrolyzer 800 can be 10, 20, 30, 40, 50, 60, 70, 80, 90, or 100 MW, for example.

The O2 product stream 810 can include oxygen in a range of greater than 95, 96, 97, 98, or 99 vol % water based on a total volume of the O2 product stream 810. In aspects, the O2 product stream 810 is pure oxygen, e.g., contains 100 vol % oxygen. In other aspects, the O2 product stream 810 is not pure oxygen. In these aspects, the O2 product stream 810 can contain hydrogen in a range of from about 0.01 vol % to about 5 vol %; alternatively, from about 0.01 vol % to about 2 vol %; alternatively, from about 0.01 to about 1 vol %, based on a total volume of the O2 product stream 810.

The hydrogen product stream 820 can include hydrogen in a range of from about 95 vol % to about 100 vol %; alternatively, from about 98 vol % to about 100 vol % based on a total volume of the hydrogen product stream 820. In aspects, the hydrogen product stream 820 is pure hydrogen, e.g., contains 100 vol % hydrogen. In other aspects, the hydrogen product stream 820 is not pure hydrogen. In these aspects, the hydrogen product stream 820 can contain oxygen in a range of from about 0.01 vol % to about 5 vol %; alternatively, from about 0.01 vol % to about 2 vol %; alternatively, from about 0.01 to about 1 vol %, based on a total volume of the hydrogen product stream 820.

The fuel gas stream 140 can include gaseous components resulting from the combination of air stream 144, methane fuel stream 410, and O2 product stream 810. In system 5000, the methane fuel fired furnace 100 can be fluidly connected to the O2 product stream 810. In FIG. 5, the O2 product stream 810 combines with the methane fuel stream 410 to form combined stream 812. Combined stream 812 then combines with air stream 144 to form fuel gas stream 140 that feeds to the methane fuel fired furnace 100. In aspects, the order of combination of streams 410, 810, and 144 can be different than illustrated in FIG. 5. For example, the streams 410, 810, and 144 can combine at a single junction point to form the fuel gas stream 140.

The fuel gas stream 540 can include gaseous components resulting from the combination of air stream 544, hydrogen product stream 420, and hydrogen product stream 820. In system 5000, the hydrogen fired furnace 501 can be fluidly connected to the hydrogen product stream 820. In FIG. 5, the hydrogen product stream 820 combines with the hydrogen product stream 420 to form combined stream 822. Combined stream 822 then combines with air stream 544 to form fuel gas stream 540 that feeds to the hydrogen fired furnace 501. In aspects, the order of combination of streams 420, 820, and 544 can be different than illustrated in FIG. 5. For example, the streams 420, 820, and 544 can combine at a single junction point to form the fuel gas stream 540.

In aspects, the hydrocarbon cracking system 5000 can include additional methane fuel fired furnaces, each configured to receive the hydrocarbon feed stream 130 and fuel gas stream 140, each configured to produce flue gas stream 142 and hydrocarbon product stream 132, where each additional flue gas stream 142 additionally fluidly couples to the carbon capture process 200, and where each additional hydrocarbon product stream 132 additionally fluidly couples to the separator 300.

In aspects, the hydrocarbon cracking system 5000 can include additional hydrogen fired furnaces, each configured to receive the hydrocarbon feed stream 530 and fuel gas stream 540, each configured to produce hydrogen combustion product stream 542 and hydrocarbon product stream 532, where each additional hydrogen combustion product stream 542 additionally fluidly couples to the carbon capture process 200, and where each additional hydrocarbon product stream 532 additionally fluidly couples to the separator 300.

A method performed using system 5000 can include separating the hydrogen combustion product stream 542 into the water stream 722 and the residual gas stream 724, and separating the water stream 722 into the O2 product stream 810 and a hydrogen fuel stream (e.g., the hydrogen product stream 820) using the electrolyzer 800.

In aspects, the method performed using system 5000 can also include feeding the O2 product stream 810 and air (via air stream 144) to the methane fuel fired furnace 100, and feeding the hydrogen product stream 820 to the hydrogen fired furnace 501 or to another unit such as i) a hydrogen fired boiler, ii) a hydrogen sales gas pipeline, iii) a storage container, iv) another process, or v) combinations thereof.

In aspects, the method performed using system 5000 can also include separating the hydrogen-rich hydrocarbon fuel stream 310 into the methane fuel stream 410 and the hydrogen product stream 420, combusting the methane fuel stream 410 in the methane fuel fired furnace 100 to produce the flue gas stream 142, and combusting the hydrogen product stream 420 in the hydrogen fired furnace 501 to produce the hydrogen combustion product stream 542.

The method performed with system 5000 can also include feeding the flue gas stream 142 to the carbon capture process 200, and separating the flue gas stream 142 into the CO2 product stream 210 and the residual gas stream 220.

The method performed with system 5000 can also include cracking, in the methane fuel fired furnace 100, the hydrocarbon feed stream 130 to produce the hydrocarbon product stream 132 by combusting the methane fuel stream 410; and cracking, in the hydrogen fired furnace 501, the hydrocarbon feed stream 530 to produce the hydrocarbon product stream 532 by combusting the hydrogen product stream 420.

The method performed with system 5000 can also include receiving the hydrocarbon product stream 132 and the hydrocarbon product stream 532 into the separator 300, and separating the hydrocarbon product stream 132 and the hydrocarbon product stream 532 in the separator 300 to produce the hydrogen-rich hydrocarbon fuel stream 310 and the hydrocarbon product stream 320.

In aspects, the method performed using system 5000 can also include feeding the methane fuel stream 410, the O2 product stream 810, and air (via air stream 144) to the methane fuel fired furnace 100, and feeding the hydrogen product stream 420, the hydrogen product stream 820, and air (via air stream 544) to the hydrogen fired furnace 501. In alternative aspects, the method performed using system 5000 can also include feeding the methane fuel stream 410, the O2 product stream 810, and air (via air stream 144) to the methane fuel fired furnace 100, feeding the hydrogen product stream 420 and air (via air stream 544) to the hydrogen fired furnace 501, and feeding the hydrogen product stream 820 to another unit such as i) a hydrogen fired boiler, ii) a hydrogen sales gas pipeline, iii) a storage container, iv) another process, or v) combinations thereof. In other alternative aspects, the method performed using system 5000 can also include feeding the methane fuel stream 410, the O2 product stream 810, and air (via air stream 144) to the methane fuel fired furnace 100, feeding the hydrogen product stream 820 and air (via air stream 544) to the hydrogen fired furnace 501, and feeding the hydrogen product stream 420 to another unit such as i) a hydrogen fired boiler, ii) a hydrogen sales gas pipeline, iii) a storage container, iv) another process, or v) combinations thereof.

The streams in the method performed with system 5000 can have any composition described herein. For example, the hydrogen-rich hydrocarbon fuel stream 310 can include hydrogen, methane, and other light hydrocarbons. Hydrogen can be present in the hydrogen-rich hydrocarbon fuel stream 310 in a range of from about 40 vol % to about 100 vol %; alternatively, in a range of from about 40 vol % to about 90 vol %; alternatively, in a range of from about 90 vol % to about 100 vol % based on a total volume of the hydrogen-rich hydrocarbon fuel stream 310. Additionally, the flue gas stream 142 can include carbon dioxide, nitrogen, and water vapor. Additionally, the hydrogen combustion product stream 542 can include nitrogen, uncombusted oxygen, and water vapor, and can be free of carbon dioxide. In aspects of the method performed with system 5000, the hydrogen product stream 420 is not feed to the methane fuel fired furnace 100. In additional or alternative aspects of the method performed with system 5000, the hydrogen product stream 420 is not connected to, and does not flow to, the carbon capture process 200.

FIG. 6 is a schematic diagram of an alternative embodiment of a hydrocarbon cracking system 6000. The system 6000 in FIG. 6 includes the configuration of equipment as described for the system 3000 in FIG. 3, and additionally includes a separator 700 and electrolyzer 800 described for FIG. 5.

Similar to above descriptions, the hydrocarbon cracking system 6000 includes the splitter 430, which is fluidly connected to the hydrogen product stream 420 and is configured to split the hydrogen product stream 420 into a first stream 422 and a second stream 424.

The fuel gas stream 540 can include gaseous components resulting from the combination of air stream 544, the first stream 422, and hydrogen product stream 820. In FIG. 6, the first stream 422 is configured to combine with the hydrogen product stream 820 to form combined stream 822. Combined stream 822 is configured to combine with air stream 544 to form the fuel gas stream 540 that is introduced to the hydrogen fired furnace 501. In aspects, the order of combination of streams 422, 820, and 544 can be different than illustrated in FIG. 6. For example, the streams 422, 820, and 544 can combine at a single junction point to form the fuel gas stream 540.

In aspects, the hydrocarbon cracking system 6000 can include additional methane fuel fired furnaces, each configured to receive the hydrocarbon feed stream 130 and fuel gas stream 140, each configured to produce flue gas stream 142 and hydrocarbon product stream 132, where each additional flue gas stream 142 additionally fluidly couples to the carbon capture process 200, and where each additional hydrocarbon product stream 132 additionally fluidly couples to the separator 300.

In aspects, the hydrocarbon cracking system 6000 can include additional hydrogen fired furnaces, each configured to receive the hydrocarbon feed stream 530 and fuel gas stream 540, each configured to produce hydrogen combustion product stream 542 and hydrocarbon product stream 532, where each additional hydrogen combustion product stream 542 additionally fluidly couples to the carbon capture process 200, and where each additional hydrocarbon product stream 532 additionally fluidly couples to the separator 300.

A method performed using system 6000 can include separating the hydrogen combustion product stream 542 into the water stream 722 and the residual gas stream 724, and separating the water stream 722 into the O2 product stream 810 and a hydrogen fuel stream (e.g., the hydrogen product stream 820) using the electrolyzer 800.

In aspects, the method performed using system 6000 can also include feeding the O2 product stream 810 and air (via air stream 144) to the methane fuel fired furnace 100, and feeding the hydrogen product stream 820 to the hydrogen fired furnace 501 or to another unit such as i) a hydrogen fired boiler, ii) a hydrogen sales gas pipeline, iii) a storage container, iv) another process, or v) combinations thereof.

The method performed with system 6000 can include separating the hydrogen-rich hydrocarbon fuel stream 310 into the methane fuel stream 410 and the hydrogen product stream 420, combusting the methane fuel stream 410 in the methane fuel fired furnace 100 to produce the flue gas stream 142.

The method performed with system 6000 can also include feeding the flue gas stream 142 to the carbon capture process 200.

The method performed with system 6000 can also include separating the flue gas stream 142 into the CO2 product stream 210 and the residual gas stream 220.

The method performed with system 6000 can also include cracking, in the methane fuel fired furnace 100, the hydrocarbon feed stream 130 to produce the hydrocarbon product stream 132 by combusting the methane fuel stream 410.

The method performed with system 6000 can also include receiving the hydrocarbon product stream 132 and the hydrocarbon product stream 532 into the separator 300, and separating the hydrocarbon product stream 132 and the hydrocarbon product stream 532 in the separator 300 to produce the hydrogen-rich hydrocarbon fuel stream 310 and the hydrocarbon product stream 320.

The method performed with system 6000 can also include splitting the hydrogen product stream 420 into a first stream 422 and a second stream 424, and simultaneously flowing hydrogen product in the first stream 422 and in the second stream 424. Alternatively, the method can include switching or selectively directing (e.g., by splitter 430) a flow of the hydrogen product stream 420: 1) from flowing to both the hydrogen fired furnace 501 via stream 422 and to the unit 600 via stream 424, 2) from flowing only to the hydrogen fired furnace 501 via stream 422 to only to the unit 600 via stream 424, and 3) from flowing only to the hydrogen fired furnace 501 via stream 422 to flow to both the furnace 501 via stream 422 and to the unit 600 via stream 424. In aspects where hydrogen product flows via second stream 424 only to the unit 600, the method can additionally include selectively directing a flow of hydrogen product stream 420: 1) from flowing only to the unit 600 via stream 424 to only to the hydrogen fired furnace 501 via stream 422, or 2) from flowing only to the unit 600 via stream 424 to flow to both the furnace 501 via stream 422 and to the unit 600 via stream 424. Selectively directing can include actuating a valve from a closed position to an open position and actuating another valve from an open position to a closed position.

In aspects where hydrogen product flows via first stream 422, the method can include combusting the first stream 422 of the hydrogen product stream 420 and the hydrogen product stream 820 in the hydrogen fired furnace 501 to produce the hydrogen combustion product stream 542; and cracking, in the hydrogen fired furnace 501, the hydrocarbon feed stream 530 to produce the hydrocarbon product stream 532 by combusting the first stream 422 of the hydrogen product stream 420 and the hydrogen product stream 820.

In aspects where hydrogen product flows via second stream 424, the method can include feeding the second stream 424 to i) another hydrogen fired furnace, ii) a hydrogen fired boiler, iii) a sales gas pipeline, iv) a storage container, v) another process, or vi) a combination thereof.

In aspects, the method performed using system 6000 can also include feeding the methane fuel stream 410, the O2 product stream 810, and air (via air stream 144) to the methane fuel fired furnace 100, and feeding the first stream 422 of the hydrogen product stream 420, the hydrogen product stream 820, and air (via air stream 544) to the hydrogen fired furnace 501. In alternative aspects, the method performed using system 6000 can also include feeding the methane fuel stream 410, the O2 product stream 810, and air (via air stream 144) to the methane fuel fired furnace 100, feeding the first stream 422 of the hydrogen product stream 420 and air (via air stream 544) to the hydrogen fired furnace 501, and feeding the hydrogen product stream 820 to another unit such as i) a hydrogen fired boiler, ii) a hydrogen sales gas pipeline, iii) a storage container, iv) another process, or v) combinations thereof. In other alternative aspects, the method performed using system 6000 can also include feeding the methane fuel stream 410, the O2 product stream 810, and air (via air stream 144) to the methane fuel fired furnace 100, feeding the hydrogen product stream 820 and air (via air stream 544) to the hydrogen fired furnace 501, and feeding the first stream 422 of the hydrogen product stream 420 to another unit such as i) a hydrogen fired boiler, ii) a hydrogen sales gas pipeline, iii) a storage container, iv) another process, or v) combinations thereof.

The streams in the method performed with system 6000 can have any composition described herein. For example, the hydrogen-rich hydrocarbon fuel stream 310 can include hydrogen, methane, and other light hydrocarbons. Hydrogen can be present in the hydrogen-rich hydrocarbon fuel stream 310 in a range of from about 40 vol % to about 100 vol %; alternatively, in a range of from about 40 vol % to about 90 vol %; alternatively, in a range of from about 90 vol % to about 100 vol % based on a total volume of the hydrogen-rich hydrocarbon fuel stream 310. Additionally, the flue gas stream 142 can include carbon dioxide, nitrogen, and water vapor. Additionally, the hydrogen combustion product stream 542 can include nitrogen, uncombusted oxygen, and water vapor, and can be free of carbon dioxide. In aspects of the method performed with system 6000, none of the hydrogen product stream 420, the first stream 422 of the hydrogen product stream 420, and the second stream 424 of the hydrogen product stream 420 is fed to the methane fuel fired furnace 100. In additional or alternative aspects of the method performed with system 6000, none of the hydrogen product stream 420, the first stream 422 of the hydrogen product stream 420, and the second stream 424 of the hydrogen product stream 420 is connected to, and none flow to, the carbon capture process 200.

FIG. 7 is a schematic diagram of an alternative embodiment of a hydrocarbon cracking system 7000. The system 7000 in FIG. 7 includes the configuration of equipment as described for the system 4000 in FIG. 4, and additionally includes a separator 700 and electrolyzer 800 described for FIG. 5.

Similar to above descriptions, the hydrocarbon cracking system 7000 includes the splitter 430, which is fluidly connected to the hydrogen product stream 420 and is configured to split the hydrogen product stream 420 into a first stream 422 and a second stream 424.

The fuel gas stream 540 can include gaseous components resulting from the combination of air stream 544, the first stream 422, and hydrogen product stream 820. In FIG. 7, the first stream 422 is configured to combine with the hydrogen product stream 820 to form combined stream 822. Combined stream 822 is configured to combine with air stream 544 to form the fuel gas stream 540 that is introduced to the hydrogen fired furnace 501. In aspects, the order of combination of streams 422, 820, and 544 can be different than illustrated in FIG. 6. For example, the streams 422, 820, and 544 can combine at a single junction point to form the fuel gas stream 540.

The fuel gas stream 140 can include gaseous components resulting from the combination of air stream 144, methane fuel stream 410, O2 product stream 810, and second stream 424. In FIG. 7, the second stream 424 of the hydrogen product stream 420 is configured to combine with the methane fuel stream 410 to form a combined stream 418. The combined stream 418 is configured to combine with O2 product stream 810 to form combined stream 812. Combined stream 812 is configured to combine with the air stream 144 to form the fuel gas stream 140. In aspects, the order of combination of streams 410, 424, 810, and 144 can be different than illustrated in FIG. 7. For example, the streams 410, 424, 810, and 144 can combine at a single junction point to form the fuel gas stream 140.

The configuration in FIG. 7 can be useful for directing hydrogen product to the methane fuel fired furnace 100. For example, in a scenario where the flow of hydrogen in the hydrogen product stream 420 is greater than the capacity of the hydrogen fired furnace 501, excess hydrogen product can be routed to the methane fuel fired furnace 100 via second stream 424 and splitter 430 (in addition to flow of hydrogen product continuing in first stream 422). In another example, in a scenario where the hydrogen fired furnace 501 is taken offline, the flow of hydrogen in the hydrogen product stream 420 can be directed to flow only in second stream 424 to the methane fuel fired furnace 100.

In aspects, the hydrocarbon cracking system 7000 can include additional methane fuel fired furnaces, each configured to receive the hydrocarbon feed stream 130 and fuel gas stream 140, each configured to produce flue gas stream 142 and hydrocarbon product stream 132, where each additional flue gas stream 142 additionally fluidly couples to the carbon capture process 200, and where each additional hydrocarbon product stream 132 additionally fluidly couples to the separator 300.

In aspects, the hydrocarbon cracking system 7000 can include additional hydrogen fired furnaces, each configured to receive the hydrocarbon feed stream 530 and fuel gas stream 540, each configured to produce hydrogen combustion product stream 542 and hydrocarbon product stream 532, where each additional hydrogen combustion product stream 542 additionally fluidly couples to the carbon capture process 200, and where each additional hydrocarbon product stream 532 additionally fluidly couples to the separator 300.

A method performed using system 7000 can include separating the hydrogen combustion product stream 542 into the water stream 722 and the residual gas stream 724, and separating the water stream 722 into the O2 product stream 810 and a hydrogen fuel stream (e.g., the hydrogen product stream 820) using the electrolyzer 800. In aspects, the method performed using system 7000 can also include feeding the O2 product stream 810 and air (via air stream 144) to the methane fuel fired furnace 100, and feeding the hydrogen product stream 820 to the hydrogen fired furnace 501 or to another unit such as i) a hydrogen fired boiler, ii) a hydrogen sales gas pipeline, iii) a storage container, iv) another process, or v) combinations thereof.

The method performed with system 7000 can include separating the hydrogen-rich hydrocarbon fuel stream 310 into the methane fuel stream 410 and the hydrogen product stream 420, combusting the methane fuel stream 410 in the methane fuel fired furnace 100 to produce the flue gas stream 142.

The method performed with system 7000 can also include feeding the flue gas stream 142 to the carbon capture process 200.

The method performed with system 7000 can also include separating the flue gas stream 142 into the CO2 product stream 210 and the residual gas stream 220.

The method performed with system 7000 can also include cracking, in the methane fuel fired furnace 100, the hydrocarbon feed stream 130 to produce the hydrocarbon product stream 132 by combusting the methane fuel stream 410.

The method performed with system 7000 can also include receiving the hydrocarbon product stream 132 and the hydrocarbon product stream 532 into the separator 300, and separating the hydrocarbon product stream 132 and the hydrocarbon product stream 532 in the separator 300 to produce the hydrogen-rich hydrocarbon fuel stream 310 and the hydrocarbon product stream 320.

The method performed with system 7000 can also include splitting the hydrogen product stream 420 into a first stream 422 and a second stream 424, and simultaneously flowing hydrogen product in the first stream 422 and in the second stream 424. Alternatively, the method can include switching or selectively directing (e.g., by splitter 430) a flow of the hydrogen product stream 420: 1) from flowing to both the hydrogen fired furnace 501 via stream 422 and to the methane fuel fired furnace 100 via stream 424, 2) from flowing only to the hydrogen fired furnace 501 via stream 422 to only to the methane fuel fired furnace 100 via stream 424, and 3) from flowing only to the hydrogen fired furnace 501 via stream 422 to flow to both the hydrogen fired furnace 501 via stream 422 and to the methane fuel fired furnace 100 via stream 424. In aspects where hydrogen product flows via second stream 424 only to the methane fuel fired furnace 100, the method can additionally include selectively directing a flow of hydrogen product stream 420: 1) from flowing only to the methane fuel fired furnace 100 via stream 424 to only to the hydrogen fired furnace 501 via stream 422, or 2) from flowing only to the methane fuel fired furnace 100 via stream 424 to flow to both the furnace 501 via stream 422 and to the methane fuel fired furnace 100 via stream 424. Selectively directing can include actuating a valve from a closed position to an open position and actuating another valve from an open position to a closed position.

In aspects where hydrogen product flows via first stream 422, the method can include combusting the first stream 422 of the hydrogen product stream 420 and the hydrogen product stream 820 in the hydrogen fired furnace 501 to produce the hydrogen combustion product stream 542; and cracking, in the hydrogen fired furnace 501, the hydrocarbon feed stream 530 to produce the hydrocarbon product stream 532 by combusting the first stream 422 of the hydrogen product stream 420 and the hydrogen product stream 820.

In aspects where hydrogen product flows via second stream 424, the method can include feeding the second stream 424 to the methane fuel fired furnace 100.

In aspects, the method performed using system 7000 can also include feeding the methane fuel stream 410, the second stream 424 of the hydrogen product stream 420, and the O2 product stream 810, and air (via air stream 144) to the methane fuel fired furnace 100; and feeding the first stream 422 of the hydrogen product stream 420, the hydrogen product stream 820, and air (via air stream 544) to the hydrogen fired furnace 501. In alternative aspects, the method performed using system 7000 can also include feeding the methane fuel stream 410, the second stream 424 of the hydrogen product stream 420, the O2 product stream 810, and air (via air stream 144) to the methane fuel fired furnace 100, feeding the first stream 422 of the hydrogen product stream 420 and air (via air stream 544) to the hydrogen fired furnace 501, and feeding the hydrogen product stream 820 to another unit such as i) a hydrogen fired boiler, ii) a hydrogen sales gas pipeline, iii) a storage container, iv) another process, or v) combinations thereof. In other alternative aspects, the method performed using system 7000 can also include feeding the methane fuel stream 410, the second stream 424 of the hydrogen product stream 420, the O2 product stream 810, and air (via air stream 144) to the methane fuel fired furnace 100, feeding the hydrogen product stream 820 and air (via air stream 544) to the hydrogen fired furnace 501, and feeding the first stream 422 of the hydrogen product stream 420 to another unit such as i) a hydrogen fired boiler, ii) a hydrogen sales gas pipeline, iii) a storage container, iv) another process, or v) combinations thereof.

The method performed with system 7000 can also include switching (e.g., by splitter 430) a flow of the hydrogen product stream 420 from only to the hydrogen fired furnace 501 via stream 422 to only to the methane fuel fired furnace 100 via stream 424. Alternatively, the method performed with system 7000 can also include switching a flow of the hydrogen product stream 420 from only to the hydrogen fired furnace 501 via stream 422 to flow to both the hydrogen fired furnace 501 via stream 422 and to the methane fuel fired furnace 100 via stream 424. Alternatively, the method performed with system 7000 can also include switching a flow of the hydrogen product stream 420 from only to the unit via stream 424 to flow to both the hydrogen fired furnace 501 via stream 422 and to the methane fuel fired furnace 100 via stream 424.

The streams in the method performed with system 7000 can have any composition described herein. For example, the hydrogen-rich hydrocarbon fuel stream 310 can include hydrogen, methane, and other light hydrocarbons. Hydrogen can be present in the hydrogen-rich hydrocarbon fuel stream 310 in a range of from about 40 vol % to about 100 vol %; alternatively, in a range of from about 40 vol % to about 90 vol %; alternatively, in a range of from about 90 vol % to about 100 vol % based on a total volume of the hydrogen-rich hydrocarbon fuel stream 310. Additionally, the flue gas stream 142 can include carbon dioxide, nitrogen, and water vapor. Additionally, the hydrogen combustion product stream 542 can include nitrogen, uncombusted oxygen, and water vapor, and can be free of carbon dioxide. In aspects of the method performed with system 7000, none of the hydrogen product stream 420, the first stream 422 of the hydrogen product stream 420, and the second stream 424 of the hydrogen product stream 420 is fed to the methane fuel fired furnace 100. In additional or alternative aspects of the method performed with system 7000, none of the hydrogen product stream 420, the first stream 422 of the hydrogen product stream 420, and the second stream 424 of the hydrogen product stream 420 is connected to, and none flow to, the carbon capture process 200.

### Additional Description

Accordingly, the scope of protection is not limited by the description set out above but is only limited by the claims which follow, that scope including all equivalents of the subject matter of the claims. Each and every claim is incorporated into the specification as an embodiment of the present disclosure. Thus, the claims are a further description and are an addition to the detailed description of the present disclosure. The disclosures of all patents, patent applications, and publications cited herein are hereby incorporated by reference.

Aspects of processes and systems have been described. The following are non-limiting, specific aspects in accordance with the present disclosure:

In Aspect 1, the techniques described herein relate to a process including: separating a hydrogen-rich hydrocarbon fuel stream into a hydrogen product stream and a methane fuel stream, wherein the hydrogen-rich hydrocarbon fuel stream includes hydrogen, methane, and other light hydrocarbons, wherein hydrogen is present in the hydrogen-rich hydrocarbon fuel stream in a range of from about 40 vol % to about 100 vol %; alternatively, in a range of from about 40 vol % to about 90 vol %; alternatively, in a range of from about 90 vol % to about 100 vol % based on a total volume of the hydrogen-rich hydrocarbon fuel stream; combusting the methane fuel stream in a methane fuel fired furnace to produce a flue gas stream including carbon dioxide, nitrogen, and water vapor; and feeding the flue gas stream to a carbon capture process.

In Aspect 2, the techniques described herein relate to the process of Aspect 1, further including: cracking, in the methane fuel fired furnace, a hydrocarbon feed stream to produce a hydrocarbon product stream by combusting the methane fuel stream.

In Aspect 3, the techniques described herein relate to the process of Aspect 2, further including: receiving the hydrocarbon product stream into a separator; and separating, in the separator, the hydrocarbon product stream into the hydrogen-rich hydrocarbon fuel stream and a second hydrocarbon product stream.

In Aspect 4, the techniques described herein relate to the process of any of Aspects 1 to 3, wherein the hydrogen product stream is not fed to the methane fuel fired furnace, wherein the hydrogen product stream is not fed to the carbon capture process, or both.

In Aspect 5, the techniques described herein relate to the process of any of Aspects 1 to 4, further including: feeding the hydrogen product stream to i) a hydrogen fired furnace, ii) a hydrogen fired boiler, iii) a hydrogen sales gas pipeline, iv) a storage container, or v) another process.

In Aspect 6, the techniques described herein relate to the process of Aspect 1, further including: feeding the hydrogen product stream to a hydrogen fired furnace; and combusting the hydrogen product stream in the hydrogen fired furnace to produce a hydrogen combustion product stream including water, nitrogen and uncombusted oxygen, wherein the hydrogen combustion product stream is free of carbon dioxide and is not fed to the carbon capture process.

In Aspect 7, the techniques described herein relate to the process of Aspect 6, further including: cracking, in the methane fuel fired furnace, a first hydrocarbon feed stream to produce a first hydrocarbon product stream by combusting the methane fuel stream; and cracking, in the hydrogen fired furnace, a second hydrocarbon feed stream to produce a second hydrocarbon product stream by combusting the hydrogen product stream.

In Aspect 8, the techniques described herein relate to the process of Aspect 7, further including: receiving the first hydrocarbon product stream and the second hydrocarbon product stream into a separator; and separating, in the separator, the first hydrocarbon product stream and the second hydrocarbon product stream into the hydrogen-rich hydrocarbon fuel stream and a third hydrocarbon product stream.

In Aspect 9, the techniques described herein relate to the process of any Aspects 6 to 8, further including: separating the hydrogen combustion product stream into a water stream and a residual gas stream; separating the water stream into a hydrogen fuel stream and an O2 product stream using an electrolyzer; feeding the O2 product stream and air to the methane fuel fired furnace; and feeding the hydrogen fuel stream to the hydrogen fired furnace.

In Aspect 10, the techniques described herein relate to the process of any Aspects 6 to 9, i) wherein the hydrogen product stream is not fed to the methane fuel fired furnace, ii) wherein the hydrogen product stream is not fed to the carbon capture process, or both, iii) wherein the hydrogen combustion product stream is not fed to the carbon capture process, or iv) combinations of i), ii), and iii).

In Aspect 11, the techniques described herein relate to the process of Aspect 1, further including: splitting the hydrogen product stream into a first stream and a second stream; feeding the first stream to a hydrogen fired furnace; and feeding the second stream to i) another hydrogen fired furnace, ii) a hydrogen fired boiler, iii) a sales gas pipeline, iv) a storage container, v) another process, or vi) a combination thereof; and combusting the first stream of the hydrogen product stream in the hydrogen fired furnace to produce a hydrogen combustion product stream including water, nitrogen and uncombusted oxygen, wherein the hydrogen combustion product stream is free of carbon dioxide and is not fed to the carbon capture process.

In Aspect 12, the techniques described herein relate to the process of Aspect 11, further including: cracking, in the hydrogen fired furnace, a first hydrocarbon feed stream to produce a first hydrocarbon product stream by combusting the first stream of the hydrogen product stream; and cracking, in the methane fuel fired furnace, a second hydrocarbon feed stream to produce a second hydrocarbon product stream by combusting the methane fuel stream.

In Aspect 13, the techniques described herein relate to the process of Aspect 12, further including: receiving the first hydrocarbon product stream and the second hydrocarbon product stream into a separator; and separating, in the separator, the first hydrocarbon product stream and the second hydrocarbon product stream into the hydrogen-rich hydrocarbon fuel stream and a third hydrocarbon product stream.

In Aspect 14, the techniques described herein relate to the process of any Aspects 11 to 13, further including: separating the hydrogen combustion product stream into a water stream and a residual gas stream; separating the water stream into a hydrogen fuel stream and an O2 product stream using an electrolyzer; feeding the O2 product stream to the methane fuel fired furnace; and feeding the hydrogen fuel stream to the hydrogen fired furnace.

In Aspect 15, the techniques described herein relate to the process of any of Aspects 11 to 14, i) wherein the hydrogen product stream is not fed to the methane fuel fired furnace, ii) wherein the hydrogen product stream is not fed to the carbon capture process, or both, iii) wherein the hydrogen combustion product stream is not fed to the carbon capture process, or iv) combinations of i), ii), and iii).

In Aspect 16, the techniques described herein relate to the process of Aspect 1, further including: splitting the hydrogen product stream into a first stream and a second stream; feeding the first stream to a hydrogen fired furnace; combusting the first stream of the hydrogen product stream in the hydrogen fired furnace to produce a hydrogen combustion product stream; feeding the second stream to the methane fuel fired furnace; and combusting the second stream of the hydrogen product stream in the methane fuel fired furnace to produce a flue gas stream.

In Aspect 17, the techniques described herein relate to the process of Aspect 16, further including: cracking, in the hydrogen fired furnace, a first hydrocarbon feed stream to produce a first hydrocarbon product stream by combusting the first stream of the hydrogen product stream; and cracking, in the methane fuel fired furnace, a second hydrocarbon feed stream to produce a second hydrocarbon product stream by combusting the second stream of the hydrogen product stream and the methane fuel stream.

In Aspect 18, the techniques described herein relate to the process of Aspect 17, further including: receiving the first hydrocarbon product stream and the second hydrocarbon product stream into a separator; and separating, in the separator, the first hydrocarbon product stream and the second hydrocarbon product stream into the hydrogen-rich hydrocarbon fuel stream and a third hydrocarbon product stream.

In Aspect 19, the techniques described herein relate to the process of any of Aspects 16 to 18, further including: separating the hydrogen combustion product stream into a water stream and a residual gas stream; separating the water stream into a hydrogen fuel stream and an O2 product stream using an electrolyzer; feeding the O2 product stream to the methane fuel fired furnace; and feeding the hydrogen fuel stream to the hydrogen fired furnace.

In Aspect 20, the techniques described herein relate to the process of any of Aspects 16 to 19, i) wherein the hydrogen product stream is not fed to the methane fuel fired furnace, ii) wherein the hydrogen product stream is not fed to the carbon capture process, or both, iii) wherein the hydrogen combustion product stream is not fed to the carbon capture process, or iv) combinations of i), ii), and iii).

In Aspect 21, the techniques described herein relate to the process of any of Aspects 1 to 20, wherein the hydrogen-rich hydrocarbon fuel stream includes methane in a range of from about 10 vol % to about 85 vol % based on a total volume of the hydrogen-rich hydrocarbon fuel stream.

In Aspect 22, the techniques described herein relate to the process of any of Aspects 1 to 21, wherein the hydrogen product stream includes hydrogen in a range of from about 88 vol % to about 99.9 vol % based on a total volume of the hydrogen product stream.

In Aspect 23, the techniques described herein relate to the process of any of Aspects 1 to 22, wherein the methane fuel stream includes methane in a range of from about 60 vol % to about 100 vol % based on a total volume of the methane fuel stream.

In Aspect 24, the techniques described herein relate to a process comprising separating a hydrogen combustion product stream into a water stream and a residual gas stream; separating the water stream into a hydrogen fuel stream and an O2 product stream using an electrolyzer; feeding the O2 product stream to a methane fuel fired furnace; and feeding the hydrogen fuel stream to a hydrogen fired furnace.

In aspect 25, the techniques described herein relate to the process of Aspect 24, further comprising separating a hydrogen-rich hydrocarbon fuel stream into a hydrogen product stream and a methane fuel stream, wherein the hydrogen-rich hydrocarbon fuel stream includes hydrogen, methane, and other light hydrocarbons, wherein hydrogen is present in the hydrogen-rich hydrocarbon fuel stream in a range of from about 40 vol % to about 100 vol %; alternatively, in a range of from about 40 vol % to about 90 vol %; alternatively, in a range of from about 90 vol % to about 100 vol % based on a total volume of the hydrogen-rich hydrocarbon fuel stream; combusting the methane fuel stream in a methane fuel fired furnace to produce a flue gas stream including carbon dioxide, nitrogen, and water vapor; and feeding the flue gas stream to a carbon capture process.

In Aspect 26, the techniques described herein relate to the process of Aspect 25, further including: splitting the hydrogen product stream into a first stream and a second stream; feeding the first stream to a hydrogen fired furnace; and feeding the second stream to i) another hydrogen fired furnace, ii) a hydrogen fired boiler, iii) a sales gas pipeline, iv) a storage container, v) another process, or vi) a combination thereof; and combusting the first stream of the hydrogen product stream in the hydrogen fired furnace to produce a hydrogen combustion product stream including water, nitrogen and uncombusted oxygen, wherein the hydrogen combustion product stream is free of carbon dioxide and is not fed to the carbon capture process.

In Aspect 27, the techniques described herein relate to the process of Aspect 25, further including: splitting the hydrogen product stream into a first stream and a second stream; feeding the first stream to a hydrogen fired furnace; and feeding the second stream to i) another hydrogen fired furnace, ii) a hydrogen fired boiler, iii) a sales gas pipeline, iv) a storage container, v) another process, or vi) a combination thereof; and combusting the first stream of the hydrogen product stream in the hydrogen fired furnace to produce a hydrogen combustion product stream including water, nitrogen and uncombusted oxygen, wherein the hydrogen combustion product stream is free of carbon dioxide and is not fed to the carbon capture process.

In Aspect 28, the techniques described herein relate to the process of Aspect 25, further including: splitting the hydrogen product stream into a first stream and a second stream; feeding the first stream to a hydrogen fired furnace; combusting the first stream of the hydrogen product stream in the hydrogen fired furnace to produce a hydrogen combustion product stream; feeding the second stream to the methane fuel fired furnace; and combusting the second stream of the hydrogen product stream in the methane fuel fired furnace to produce a flue gas stream.

In Aspect 29, the techniques described herein relate to the process of any Aspects 24 to 28, i) wherein the hydrogen product stream is not fed to the methane fuel fired furnace, ii) wherein the hydrogen product stream is not fed to the carbon capture process, or both, iii) wherein the hydrogen combustion product stream is not fed to the carbon capture process, or iv) combinations of i), ii), and iii).

In Aspect 30, the techniques described herein relate to the process of any Aspects 1 to 29, wherein separating a hydrogen-rich hydrocarbon fuel stream is performed by pressure swing absorption, membrane separation, cryogenic separation, or combinations thereof.

In Aspect 31, the techniques described herein relate to the process of any Aspects 1 to 30, wherein the carbon capture process comprises recovering a CO2 product stream from the flue gas stream by absorption, adsorption, membrane separation, carbon dioxide condensation, or a combination thereof.

In Aspect 32, the techniques described herein relate to a hydrocarbon cracking system including: a methane fuel fired furnace fluidly connected to a first hydrocarbon feed stream and configured to produce a first hydrocarbon product stream; a first separator fluidly connected to the first hydrocarbon product stream and configured to separate the first hydrocarbon product stream into a hydrogen-rich hydrocarbon fuel stream and a third hydrocarbon product stream; and a second separator fluidly connected to the hydrogen-rich hydrocarbon fuel stream and configured to separate the hydrogen-rich hydrocarbon fuel stream into a hydrogen product stream and a methane fuel stream; wherein the methane fuel fired furnace is configured to fluidly couple with and to receive the methane fuel stream.

In Aspect 33, the techniques described herein relate to the system of Aspect 32, further including a hydrogen fired furnace fluidly connected to a second hydrocarbon feed stream and configured to produce a second hydrocarbon product stream.

In Aspect 34, the techniques described herein relate to the system of Aspect 33, wherein the hydrogen fired furnace is configured to fluidly couple with and to receive the hydrogen product stream.

In Aspect 35, the techniques described herein relate to the system of any of Aspects 32 to 34, further including: a splitter coupled to the hydrogen product stream and configured to split the hydrogen product stream into a first stream and a second stream.

In Aspect 36, the techniques described herein relate to the system of Aspect 35, wherein the hydrogen fired furnace is configured to receive the first stream of the hydrogen product stream.

In Aspect 37, the techniques described herein relate to the system of any of Aspects 35 or 36, further including a second hydrogen fired furnace, a hydrogen fired boiler, a hydrogen sale gas pipeline, or a storage container, wherein the methane fuel fired furnace, the second hydrogen fired furnace, the hydrogen fired boiler, the hydrogen sale gas pipeline, or the storage container is configured to receive the second stream of the hydrogen product stream.

In Aspect 38, the techniques described herein relate to the system of any of Aspects 33 to 37, wherein the hydrogen fired furnace is configured to produce a hydrogen combustion product stream.

In Aspect 39, the techniques described herein relate to the system of Aspect 38, further including: a condenser fluidly coupled to the hydrogen fired furnace and configured to receive the hydrogen combustion product stream and to produce a water stream; and an electrolyzer fluidly coupled to i) the condenser via the water stream, ii) the hydrogen fired furnace, and iii) the methane fuel fired furnace, wherein the electrolyzer is configured to separate the water stream into a O2 product stream and a second hydrogen product stream, wherein the hydrogen fired furnace is configured to receive the second hydrogen product stream, wherein the methane fuel fired furnace is configured to receive the O2 product stream.

In Aspect 40, the techniques described herein relate to the system of any of Aspects 32 to 39, wherein the first separation comprises a pressure swing absorption unit, a membrane separation unit, a cryogenic separation unit, or combinations thereof.

In Aspect 41, the techniques described herein relate to the system of any of Aspects 32 to 40, further comprising a carbon capture unit having one or more vessels configured for recovering a CO2 product stream from the flue gas stream by absorption, adsorption, membrane separation, carbon dioxide condensation, or a combination thereof.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A process comprising:
separating a hydrogen-rich hydrocarbon fuel stream into a hydrogen product stream and a methane fuel stream, wherein the hydrogen-rich hydrocarbon fuel stream comprises hydrogen, methane, and other light hydrocarbons, wherein hydrogen is present in the hydrogen-rich hydrocarbon fuel stream in a range of from about 40 vol % to about 100 vol % based on a total volume of the hydrogen-rich hydrocarbon fuel stream;
combusting the methane fuel stream in a methane fuel fired furnace to produce a flue gas stream comprising carbon dioxide, nitrogen, and water vapor; and
feeding the flue gas stream to a carbon capture process.

2. The process of claim 1, further comprising:
cracking, in the methane fuel fired furnace, a hydrocarbon feed stream to produce a hydrocarbon product stream by combusting the methane fuel stream.

3. The process of claim 2, further comprising:
receiving the hydrocarbon product stream into a separator; and
separating, in the separator, the hydrocarbon product stream into the hydrogen-rich hydrocarbon fuel stream and a second hydrocarbon product stream.

4. The process of claim 1, wherein the hydrogen product stream is not fed to the methane fuel fired furnace.

5. The process of claim 1, further comprising:
feeding the hydrogen product stream to i) a hydrogen fired furnace, ii) a hydrogen fired boiler, iii) a hydrogen sales gas pipeline, iv) a storage container, or v) another process.

6. The process of claim 1, further comprising:
feeding the hydrogen product stream to a hydrogen fired furnace; and
combusting the hydrogen product stream in the hydrogen fired furnace to produce a hydrogen combustion product stream comprising water, nitrogen and uncombusted oxygen, wherein the hydrogen combustion product stream is free of carbon dioxide and is not fed to the carbon capture process.

7. The process of claim 6, further comprising:
cracking, in the methane fuel fired furnace, a first hydrocarbon feed stream to produce a first hydrocarbon product stream by combusting the methane fuel stream; and
cracking, in the hydrogen fired furnace, a second hydrocarbon feed stream to produce a second hydrocarbon product stream by combusting the hydrogen product stream.

8. The process of claim 7, further comprising:
receiving the first hydrocarbon product stream and the second hydrocarbon product stream into a separator; and
separating, in the separator, the first hydrocarbon product stream and the second hydrocarbon product stream into the hydrogen-rich hydrocarbon fuel stream and a third hydrocarbon product stream.

9. The process of claim 6, further comprising:
separating the hydrogen combustion product stream into a water stream and a residual gas stream;
separating the water stream into a hydrogen fuel stream and an O2 product stream using an electrolyzer;
feeding the O2 product stream and air to the methane fuel fired furnace; and
feeding the hydrogen fuel stream to the hydrogen fired furnace.

10. The process of claim 1, further comprising:
splitting the hydrogen product stream into a first stream and a second stream;
feeding the first stream to a hydrogen fired furnace;
feeding the second stream to i) another hydrogen fired furnace, ii) a hydrogen fired boiler, iii) a sales gas pipeline, iv) a storage container, v) another process, or vi) a combination thereof; and
combusting the first stream of the hydrogen product stream in the hydrogen fired furnace to produce a hydrogen combustion product stream comprising water, nitrogen and uncombusted oxygen, wherein the hydrogen combustion product stream is free of carbon dioxide and is not fed to the carbon capture process.

11. The process of claim 10, wherein the hydrogen combustion product stream is not fed to the carbon capture process.

12. The process of claim 10, further comprising:
cracking, in the hydrogen fired furnace, a first hydrocarbon feed stream to produce a first hydrocarbon product stream by combusting the first stream of the hydrogen product stream; and
cracking, in the methane fuel fired furnace, a second hydrocarbon feed stream to produce a second hydrocarbon product stream by combusting the methane fuel stream.

13. The process of claim 12, further comprising:
receiving the first hydrocarbon product stream and the second hydrocarbon product stream into a separator; and
separating, in the separator, the first hydrocarbon product stream and the second hydrocarbon product stream into the hydrogen-rich hydrocarbon fuel stream and a third hydrocarbon product stream.

14. The process of claim 10, further comprising:
separating the hydrogen combustion product stream into a water stream and a residual gas stream;
separating the water stream into a hydrogen fuel stream and an O2 product stream using an electrolyzer;
feeding the O2 product stream to the methane fuel fired furnace; and
feeding the hydrogen fuel stream to the hydrogen fired furnace.

15. The process of claim 1, further comprising:
splitting the hydrogen product stream into a first stream and a second stream;
feeding the first stream to a hydrogen fired furnace;
combusting the first stream of the hydrogen product stream in the hydrogen fired furnace to produce a hydrogen combustion product stream;
feeding the second stream to the methane fuel fired furnace; and
combusting the second stream of the hydrogen product stream in the methane fuel fired furnace to produce a flue gas stream.

16. The process of claim 15, wherein the hydrogen combustion product stream is not fed to the carbon capture process.

17. The process of claim 15, further comprising:
cracking, in the hydrogen fired furnace, a first hydrocarbon feed stream to produce a first hydrocarbon product stream by combusting the first stream of the hydrogen product stream; and
cracking, in the methane fuel fired furnace, a second hydrocarbon feed stream to produce a second hydrocarbon product stream by combusting the second stream of the hydrogen product stream and the methane fuel stream.

18. The process of claim 17, further comprising:
receiving the first hydrocarbon product stream and the second hydrocarbon product stream into a separator; and
separating, in the separator, the first hydrocarbon product stream and the second hydrocarbon product stream into the hydrogen-rich hydrocarbon fuel stream and a third hydrocarbon product stream.

19. The process of claim 15, further comprising:
separating the hydrogen combustion product stream into a water stream and a residual gas stream;
separating the water stream into a hydrogen fuel stream and an O2 product stream using an electrolyzer;
feeding the O2 product stream to the methane fuel fired furnace; and
feeding the hydrogen fuel stream to the hydrogen fired furnace.

20. The process of claim 1, wherein the hydrogen-rich hydrocarbon fuel stream comprises methane in a range of from about 10 vol % to about 85 vol % based on a total volume of the hydrogen-rich hydrocarbon fuel stream, wherein the hydrogen product stream comprises hydrogen in a range of from about 88 vol % to about 99.9 vol % based on a total volume of the hydrogen product stream, and wherein the methane fuel stream comprises methane in a range of from about 60 vol % to about 100 vol % based on a total volume of the methane fuel stream.
